SEQUENCE LISTING (18 sequences)

SEQ ID NO: 1 - DNA, 1369 bp, Homo sapiens
acgctctcggggcggactcccggccctccgcgccctctcgcgcggcgatggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagaatcccggctccagagcaggaaattcagcccaccagccacctcatccccaaccccctgtaaggttccatccacccctgcgtcgagctgggaaggttccatgaagcgagtcgggtccccaacccgtgcccctgggatccgagggcccctctccaagcgcctggctttggaatgctccaggcgcgccgacgcctgtgccaccccttcctcagcctggggtttgaccacccacctgaccaggggccctacctggggaaagcctgaaggt

SEQ ID NO: 2 - protein, 385 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCKNPGSRAGNSAHQPPHPQPPVRFHPPLRRAGKVP

SEQ ID NO: 3 - DNA, 1244 bp, Homo sapiens
acgctctcggggcggactcccggccctccgcgccctctcgcgcggcgatggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagtaggcaccggccgcggctccccctggacggggcgggccctgcctgagggtgggcttttccctgggtggagcaggactcccacctaaacggggcagtactcctccctgggggcgggactcctacctggggaaagcctgaagg

SEQ ID NO: 4 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 5 - DNA, 1369 bp, Homo sapiens; identical to SEQ ID NO: 1

SEQ ID NO: 6 - protein, 385 aa, Homo sapiens; identical to SEQ ID NO: 2

SEQ ID NO: 7 - DNA, 1732 bp, Homo sapiens
gaggggcgggggctggaggcagcagcgcccccgcactccccgcgtctcgcacacttgcaccggtcgctcgcgcgcagcccggcgtcgccccacgccgcgctcgctcctccctccctcctcccgctccgtggctcccgtgctcctggcgaggctcaggcgcggagcgcgcggacgggcgcaccgacagacggccccggggacgcctcggctcgcgcctcccgggcgggctatgttgattgccccgccggggccggcccgcgggatcagcacagcccggcccgcggccccggcggccaatcgggactatgaaccggaaagcgcggcgctgcctgggccacctctttctcagcctgggcatggtctacctccggatcggtggcttctcctcagtggtagctctgggcgcaagcatcatctgtaacaagatcccaggcctggctcccagacagcgggcgatctgccagagccggcccgacgccatcatcgtcataggagaaggctcacaaatgggcctggacgagtgtcagtttcagttccgcaatggccgctggaactgctctgcactgggagagcgcaccgtcttcgggaaggagctcaaagtggggagccgggaggctgcgttcacctacgccatcattgccgccggcgtggcccacgccatcacagctgcctgtacccagggcaacctgagcgactgtggctgcgacaaagagaagcaaggccagtaccaccgggacgagggctggaagtggggtggctgctctgccgacatccgctacggcatcggcttcgccaaggtctttgtggatgcccgggagatcaagcagaatgcccggactctcatgaacttgcacaacaacgaggcaggccgaaagatcctggaggagaacatgaagctggaatgtaagtgccacggcgtgtcaggctcgtgcaccaccaagacgtgctggaccacactgccacagtttcgggagctgggctacgtgctcaaggacaagtacaacgaggccgttcacgtggagcctgtgcgtgccagccgcaacaagcggcccaccttcctgaagatcaagaagccactgtcgtaccgcaagcccatggacacggacctggtgtacatcgagaagtcgcccaactactgcgaggaggacccggtgaccggcagtgtgggcacccagggccgcgcctgcaacaagacggctccccaggccagcggctgtgacctcatgtgctgtgggcgtggctacaacacccaccagtacgcccgcgtgtggcagtgcaactgtaagttccactggtgctgctatgtcaagtgcaacacgtgcagcgagcgcacggagatgtacacgtgcaagtgagccccgtgtgcacaccaccctcccgctgcaagtcagattgctgggaggactggaccgtttccaagctgcgggctccctggcaggatgctgagcttgtcttttctgctgaggagggtacttttcctgggtttcctgcaggcatccgtgggggaaaaaaaatctctcagagccctcaactattctgttccacacccaatgctgctccaccctcccccagacacagcccaggtccctccgcggctggagcgaagccttctgcagcaggaactctggacccctgggcctcatcacagcaatatttaacaatttattctgataaaaataatattaatttatttaattaaaaagaattcttccacaaaaaaaaaaaaaaaaaa

SEQ ID NO: 8 - protein, 349 aa, Homo sapiens
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 9 - DNA, 3928 bp, Homo sapiens
ttggccgccgcggcgctcggcgaggcgctcacggcctcaggcggctgcgcctagtgcaggctgcacccgcccgccagccgcgcccgggcccccggcccgcgcttgcaggggtccacggtcgctcaaccgggcttcgaggtcgcggcccccagcctgggtcaattccccgggctcacgcggcggtggcggcggcggcatcccgggctcaccatggtggcagcgcgcgccgagtgcccgcgcgtcgccggccgcccgagccgcagcgcgggctgagcgcagtctgcccgcagccccctggcccctgcccggccctgccgggcccgcgcgtcccctccggccgccgctgtctatggcgcagcccccctccctggatcatgcacagaaactttcgcaagtggattttctacgtgtttctctgctttggcgtcctgtacgtgaagctcggagcactgtcatccgtggtggccctgggagccaacatcatctgcaacaagattcctggcctagccccgcggcagcgtgccatctgccagagtcggcccgatgccatcattgtgattggggagggggcgcagatgggcatcaacgagtgccagtaccagttccgcttcggacgctggaactgctctgccctcggcgagaagaccgtcttcgggcaagagctccgagtagggagccgtgaggctgccttcacgtacgccatcaccgcggctggcgtggcgcacgccgtcaccgctgcctgcagccaagggaacctgagcaactgcggctgcgaccgcgagaagcagggctactacaaccaagccgagggctggaagtggggcggctgctcggccgacgtgcgttacggcatcgacttctcccggcgcttcgtggacgctcgggagatcaagaagaacgcgcggcgcctcatgaacctgcataacaatgaggccggcaggaaggttctagaggaccggatgcagctggagtgcaagtgccacggcgtgtctggctcctgcaccaccaaaacctgctggaccacgctgcccaagttccgagaggtgggccacctgctgaaggagaagtacaacgcggccgtgcaggtggaggtggtgcgggccagccgtctgcggcagcccaccttcctgcgcatcaaacagctgcgcagctatcagaagcccatggagacagacctggtgtacattgagaagtcgcccaactactgcgaggaggacgcggccacgggcagcgtgggcacgcagggccgtctctgcaaccgcacgtcgcccggcgcggacggctgtgacaccatgtgctgcggccgaggctacaacacccaccagtacaccaaggtgtggcagtgcaactgcaaattccactggtgctgcttcgtcaagtgcaacacctgcagcgagcgcaccgaggtcttcacctgcaagtgaggccaggcccggaggcggccgcgggcaccctggaacccggcggcattttgcacatccactcctcaccttccctgccttggtgctgccagcagcagacatagacgggtgcagaagcggggagctccaggtgcaggagggcaccggccggggcccacgccctctgcccgcctccctggggctccttcctgccacctcctcccatcacctcctgcggcagaacagcacccgtgacccacccagagagcaaggccaggggtcttggtgctccccgacggggcccggcaagttctctttcttctctctgggaaaatgaacgtccaggacacacctgtatcccagagagcaaagtgatgaggagactgagcgtccccagccccacctggcggcatggacacagaaaagctacgccggctggcctctccagaccagttcccaggctgggtctgccgctgggccctggggcggtggggacagatgttgacacaaattatttatgttttcttagtatcagaagaggattctcggcactaacacatagccagtcctaactccgtactctgtgtcagcccatcccctagacaccctctgtttcctttcccggccccacctggccggccctctgcccctgcagagctgaggcagcctggggttgatggggaccacgcggtgcctgcaggtcctagaagtgagctgggcaggggctcttcagaccacacagccctgaccgggccttggaggagagccatggacaggctcctccatgccgtctttccttcttttgaaaatcctatcaatggctgggcgcggtggctcacacctgtaatcccagcactttgggagaccgaggcaggtggatcacctgaggtcaggagttcgagaccagcctggccaacgtggtgaaaccctgtctctactaaaaatacaaaaattagctgggcgtggtggcgtgcacctgtaatcccagctactcaggaggctgagacaggacacttgcttgaacccgggaggtggaggttgcaatgagccaagattgtgccactgtattccaacttgggtgacagagcacgactctgtctcaaaaaaagaaaagaaaataatatcaagcaaatcaaaatatcacccacgtcaggttccaggagtccttcgttccctccctcatcctgtcaccctcccggcctggcacctccagtttacagagacccccttccccctggagccagcctgacccctgggacagtgctgccgcgggggtcaggcaggtggtgagtggtcagggatgtttgtcccacttgacagatgggaactctgaggcccagagtggcacaaggacctaccagagaccctgcagctgtgactggaggcttccacctttctcctgcccagcctggggctctggacctgggagaacacatcaggatgcccggggtggcagtaggtagctcccatgccttgaggcacttggctgctccctccaccccctgacagcctcattggggcctgcactcccctcggtgcctttgaggcttcgcctagccctgtcctggtccttttaccctgaccccagtgctgagaaaatctggatccagagctggacatgtcctttctggatgctcccacccctgtctctgaggagcatgagtgacagagggaggaacttgtccgaggccagagaggatggtctgcagctggctcctggtgcacccccactgcccacggcctggcccccatttagggtgtgttctcttggaagcccgtaggaatgtgttcttgagcagccgacaagccaagggcctgggctcagcctggggcaagtgggtccctcctgggctctggccctgtggtccagcccatgtgcctctaaccatgcctggacgctctgagtctccgggccaccgtgaatgccccaccctcagcctggccccatccccttaccttccccaagtcccagaacacacctgagtccataggcccggcagcctctgcaaggaacctggttaacttatgttgttatatagcgtcgaaatgtctatagtgtctttaaattattgtgacctacactgggtaccggagagggggatggctgcggcccccatccctcatcccccatcccatcccatccctcatctcatccctcatcccatcccccatcccatccctgaccccatcccccatcccatccctcatccctattccccatacccaccccccatcccatcccccatctcccgaaccaggctcctcctgcttcaacagacccttggggcccctgatgccaccgagacactcgcactgtcccttggctgccgggcaccccactgagcccctgcccaccccaaagtctgccagcaacaggccctttgcctgggtgagacgggggtcagggtggcccagatgggggtgatggcgaccaaggtgctgcgtccccacccttctgacggcagcctcccctggaggggagtgctcggcccaataaaagctggaatcggaaaaaaaaaaaaaaaaaa

SEQ ID NO: 10 - protein, 349 aa, Homo sapiens
MHRNFRKWIFYVFLCFGVLYVKLGALSSVVALGANIICNKIPGLAPRQRAICQSRPDAIIVIGEGAQMGINECQYQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMQLECKCHGVSGSCTTKTCWTTLPKFREVGHLLKEKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 11 - DNA, 2242 bp, Homo sapiens
agtctgcccgcagccccctggcccctgcccggccctgccgggcccgcgcgtcccctccggccgccgctgtctatggcgcagcccccctccctggatcatgcacagaaactttcgcaagtggattttctacgtgtttctctgctttggcgtcctgtacgtgaagctcggagcactgtcatccgtggtggccctgggagccaacatcatctgcaacaagattcctggcctagccccgcggcagcgtgccatctgccagagtcggcccgatgccatcattgtgattggggagggggcgcagatgggcatcaacgagtgccagtaccagttccgcttcggacgctggaactgctctgccctcggcgagaagaccgtcttcgggcaagagctccgagtagggagccgtgaggctgccttcacgtacgccatcaccgcggctggcgtggcgcacgccgtcaccgctgcctgcagccaagggaacctgagcaactgcggctgcgaccgcgagaagcagggctactacaaccaagccgagggctggaagtggggcggctgctcggccgacgtgcgttacggcatcgacttctcccggcgcttcgtggacgctcgggagatcaagaagaacgcgcggcgcctcatgaacctgcataacaatgaggccggcaggaaggttctagaggaccggatgcagctggagtgcaagtgccacggcgtgtctggctcctgcaccaccaaaacctgctggaccacgctgcccaagttccgagaggtgggccacctgctgaaggagaagtacaacgcggccgtgcaggtggaggtggtgcgggccagccgtctgcggcagcccaccttcctgcgcatcaaacagctgcgcagctatcagaagcccatggagacagacctggtgtacattgagaagtcgcccaactactgcgaggaggacgcggccacgggcagcgtgggcacgcagggccgtctctgcaaccgcacgtcgcccggcgcggacggctgtgacaccatgtgctgcggccgaggctacaacacccaccagtacaccaaggtgtggcagtgcaactgcaaattccactggtgctgcttcgtcaagtgcaacacctgcagcgagcgcaccgaggtcttcacctgcaagtgaggccaggcccggaggcggccgcgggcaccctggaacccggcggcattttgcacatccactcctcaccttccctgccttggtgctgccagcagcagacatagacgggtgcagaagcggggagctccaggtgcaggagggcaccggccggggcccacgccctctgcccgcctccctggggctccttcctgccacctcctcccatcacctcctgcggcagaacagcacccgtgacccacccagagagcaaggccaggggccttggtgctccccgacggggcccggcaagttctctttcttctctctgggaaaatgaacgtccaggacacacctgtatcccagagagcaaagtgatgaggagactgagcgtccccagccccacctggcggcatggacacagaaaagctacgccggctggcctctccagaccagttcccaggctgggtctgccgctgggccctggggcggtggggacagatgttgacacaaattatttatgttttcttagtatcagaagaggattctcggcactaacacatagccagtcctaactccgtactctgtgtcagcccatcccctcgacaccctctgtttcctttcccggccccacctggccggccctctgcccctgcagagctgaggcagcctggggttgatggggaccacgcggtgcctgcaggtcctagaagtgagctgggcaggggctcttcagaccacacagccctgaccgggccttggaggagagccatggacaggctcctccatgccgtctttccttcttttgaaaatcctatcaatggctgggcgcggtggctcacacctgtaatcccagcactttgggagaccgaggcaggtggatcacctgaggtcaggagttcgagaccagcctggccaacgtggtgaaaccctgtctctactaaaaatacaaaaattagctgggcgtggtggcgtgcacctgtaatcccagctactcaggaggctgagacaggacacttgcttgaacccgggaggtggaggttgcaatgagccaagattgtgccactgtattccaacctgggtgacagagcacgactctgtctcaaaaaaaaaaaaaaaaaa

SEQ ID NO: 12 - protein, 349 aa, Homo sapiens; identical to SEQ ID NO: 10

SEQ ID NO: 13 - DNA, 1259 bp, Homo sapiens
cgagtcggagctgtgtgtcgcagccgccccgaccccccgccgatcatgcgccggcgcccctggctctccagtcccactgggctgtgagccccccactcccagcccgtcagggcctgcgcgccatgggcagcgcccaccctcgcccctggctgcggctccgaccccagccccagccgcggccagcgctctgggtgctcctgttcttcctactgctgctggctgctgccatgcccaggtcagcacccaatgacattctggacctccgcctccccccggagcccgtgctcaatgccaacacagtgtgcctaacattgccaggcctgagccggcggcagatggaggtgtgtgtgcgtcaccctgatgtggctgcctcagccatacagggcatccagatcgccatccacgaatgccaacaccaattcagggaccagcgctggaactgctcaagcctggagactcgcaacaagatcccctatgagagtcccatcttcagcagaggtttccgagagagcgcttttgcctacgccatcgcagcagctggcgtggtgcacgccgtgtccaatgcgtgtgccctgggcaaactgaaggcctgtggctgtgatgcgtcccggcgaggggacgaggaggccttccgtaggaagctgcaccgcttacaactggatgcactgcagcgtggtaagggcctgagccatggggtcccggaacacccagccctgcccacagccagcccaggcctgcaggactcctgggagtggggcggctgcagccccgacatgggcttcggggagcgcttttctaaggactttctggactcccgggagcctcacagagacatccacgcgagaatgaggcttcacaacaaccgagttgggaggcaggcagtgatggagaacatgcggcggaagtgcaagtgccacggcacgtcaggcagctgccagctcaagacgtgctggcaggtgacgcccgagttccgcaccgtgggggcgctgctgcgcagccgcttccaccgcgccacgctcatccggccgcacaaccgcaacggcggccagctggagccgggcccagcgggggcaccctcgccggctccgggcgctcccgggccgcgccgacgggccagccccgccgacctggtctacttcgaaaagtctcccgacttctgcgagcgcgagccgcgcctggactcggcgggcaccgtgggccgcctgtgcaacaagagcagcgccggctcggatggctgcggcagcatgtgctgcggccgc

SEQ ID NO: 14 - protein, 379 aa, Homo sapiens
MGSAHPRPWLRLRPQPQPRPALWVLLFFLLLLAAAMPRSAPNDILDLRLPPEPVLNANTVCLTLPGLSRRQMEVCVRHPDVAASAIQGIQIAIHECQHQFRDQRWNCSSLETRNKIPYESPIFSRGFRESAFAYAIAAAGVVHAVSNACALGKLKACGCDASRRGDEEAFRRKLHRLQLDALQRGKGLSHGVPEHPALPTASPGLQDSWEWGGCSPDMGFGERFSKDFLDSREPHRDIHARMRLHNNRVGRQAVMENMRRKCKCHGTSGSCQLKTCWQVTPEFRTVGALLRSRFHRATLIRPHNRNGGQLEPGPAGAPSPAPGAPGPRRRASPADLVYFEKSPDFCEREPRLDSAGTVGRLCNKSSAGSDGCGSMCCGR

SEQ ID NO: 15 - DNA, 2375 bp, Homo sapiens
acagtcacttactctacaggcagtggggcccgacacagacagcgccgcccccgccagccagcctcgcacgccctcggaagcgcaggctcccggcgctgcgctggagggttccccggcaccccagcctcccgtccccagcccgctgcacctccgggccccccttacccttgagaggcaccgggagttgtcgcgggggggcctcgggaaattccccggacccctgtgccaggaggtgcccggttcgcccgctcttcaccccccgccccccccgagggcggtgcccgggggtgctgccccatggagcggggaggcgggcgccgtctgctccgggagccctgacccgagtcggagctgtgtgtcgcagccgccccgaccccccgccgatcatgcgccggcgcccctggctctccagtcccactgggctgtgagccccccactcccagcccgtcagggcctgcgcgccatgggcagcgcccaccctcgcccctggctgcggctccgaccccagccccagccgcggccagcgctctgggtgctcctgttcttcctactgctgctggctgctgccatgcccaggtcagcacccaatgacattctggacctccgcctccccccggagcccgtgctcaatgccaacacagtgtgcctaacattgccaggcctgagccggcggcagatggaggtgtgtgtgcgtcaccctgatgtggctgcctcagccatacagggcatccagatcgccatccacgaatgccaacaccaattcagggaccagcgctggaactgctcaagcctggagactcgcaacaagatcccctatgagagtcccatcttcagcagaggtttccgagagagcgcttttgcctacgccatcgcagcagctggcgtggtgcacgccgtgtccaatgcgtgtgccctgggcaaactgaaggcctgtggctgtgatgcgtcccggcgaggggacgaggaggccttccgtaggaagctgcaccgcttacaactggatgcactgcagcgtggtaagggcctgagccatggggtcccggaacacccagccctgcccacagccagcccaggcctgcaggactcctgggagtggggcggctgcagccccgacatgggcttcggggagcgcttttctaaggactttctggactcccgggagcctcacagagacatccacgcgagaatgaggcttcacaacaaccgagttgggaggcaggcagtgatggagaacatgcggcggaagtgcaagtgccacggcacgtcaggcagctgccagctcaagacgtgctggcaggtgacgcccgagttccgcaccgtgggggcgctgctgcgcagccgcttccaccgcgccacgctcatccggccgcacaaccgcaacggcggccagctggagccgggcccagcgggggcaccctcgccggctccgggcgctcccgggccgcgccgacgggccagccccgccgacctggtctacttcgaaaagtctcccgacttctgcgagcgcgagccgcgcctggactcggcgggcaccgtgggccgcctgtgcaacaagagcagcgccggctcggatggctgcggcagcatgtgctgcggccgcggccacaacatcctgcgccagacgcgcagcgagcgctgccactgccgcttccactggtgctgtttcgtggtctgcgaagagtgccgcatcaccgagtgggtcagcgtctgcaagtgagcggcccggggtcccctgggccctgatcgaggtcccctcctggagcctggccctctgaggcttacggtcttggcaaggcagcatcgccttggctcttgggaagaggagattggaccacatgatcttataggaacccctcagctctgaggtctgtgatcgccggacagtccaggcctgtctgaaccccaccactcacttctgtgggctctaggactgactgggttcttcctccctccccgaagcccagacagttcagttgggctgggggttgctccacaccctaaaacaagcctcagccaggcaacccgtcagtctgtctccatcctttcaccccttccctggagatgggaggtggggaatgaatggaagctgacgggcagagagaggaggattaaaaaaaagaaatagacataactgagctgaagtaattccataaagggcccagacagcctcctccaccattcccttcatcattcatttaacaaatatttattttgcactctctttgcggcactctgggggcggtggggtgcgtgggggtggcaatgcaaggcactgaggccacagatgtgagtaagcgagacacaacacttgtcctcttggaggttacattcttgctggggggaggcatgggcaataaacaagtaaatatacaaac

SEQ ID NO: 16 - protein, 417 aa, Homo sapiens
MGSAHPRPWLRLRPQPQPRPALWVLLFFLLLLAAAMPRSAPNDILDLRLPPEPVLNANTVCLTLPGLSRRQMEVCVRHPDVAASAIQGIQIAIHECQHQFRDQRWNCSSLETRNKIPYESPIFSRGFRESAFAYAIAAAGVVHAVSNACALGKLKACGCDASRRGDEEAFRRKLHRLQLDALQRGKGLSHGVPEHPALPTASPGLQDSWEWGGCSPDMGFGERFSKDFLDSREPHRDIHARMRLHNNRVGRQAVMENMRRKCKCHGTSGSCQLKTCWQVTPEFRTVGALLRSRFHRATLIRPHNRNGGQLEPGPAGAPSPAPGAPGPRRRASPADLVYFEKSPDFCEREPRLDSAGTVGRLCNKSSAGSDGCGSMCCGRGHNILRQTRSERCHCRFHWCCFVVCEECRITEWVSVCK

SEQ ID NO: 17 - DNA, 2405 bp, Homo sapiens
cccgagccgggacagtcacttactctacaggcagtggggcccgacacagacagcgccgcccccgccagccagcctcgcacgccctcggaagcgcaggctcccggcgctgcgctggagggttccccggcaccccagcctcccgtccccagcccgctgcacctccgggccccccttacccttgagaggcaccgggagttgtcgcgggggggcctcgggaaattccccggacccctgtgccaggaggtgcccggttcgcccgctcttcaccccccgccccccccgagggcggtgcccgggggtgctgccccatggagcggggaggcgggcgccgtctgctccgggagccctgacccgagtcggagctgtgtgtcgcagccgccccgaccccccgccgatcatgcgccggcgcccctggctctccagtcccactgggctgtgagccccccactcccagcccgtcagggcctgcgcgccatgggcagcgcccaccctcgcccctggctgcggctccgaccccagccccagccgcggccagcgctctgggtgctcctgttcttcctactgctgctggctgctgccatgcccaggtcagcacccaatgacattctggacctccgcctccccccggagcccgtgctcaatgccaacacagtgtgcctaacattgccaggcctgagccggcggcagatggaggtgtgtgtgcgtcaccctgatgtggctgcctcagccatacagggcatccagatcgccatccacgaatgccaacaccaattcagggaccagcgctggaactgctcaagcctggagactcgcaacaagatcccctatgagagtcccatcttcagcagaggtttccgagagagcgcttttgcctacgccatcgcagcagctggcgtggtgcacgccgtgtccaatgcgtgtgccctgggcaaactgaaggcctgtggctgtgatgcgtcccggcgaggggacgaggaggccttccgtaggaagctgcaccgcttacaactggatgcactgcagcgtggtaagggcctgagccatggggtcccggaacacccagccctgcccacagccagcccaggcctgcaggactcctgggagtggggcggctgcagccccgacatgggcttcggggagcgcttttctaaggactttctggactcccgggagcctcacagagacatccacgcgagaatgaggcttcacaacaaccgagttgggaggcaggcagtgatggagaacatgcggcggaagtgcaagtgccacggcacgtcaggcagctgccagctcaagacgtgctggcaggtgacgcccgagttccgcaccgtgggggcgctgctgcgcagccgcttccaccgcgccacgctcatccggccgcacaaccgcaacggcggccagctggagccgggcccagcgggggcaccctcgccggctccgggcgctcccgggccgcgccgacgggccagccccgccgacctggtctacttcgaaaagtctcccgacttctgcgagcgcgagccgcgcctggactcggcgggcaccgtgggccgcctgtgcaacaagagcagcgccggctcggatggctgcggcagcatgtgctgcggccgcggccacaacatcctgcgccagacgcgcagcgagcgctgccactgccgcttccactggtgctgtttcgtggtctgcgaagagtgccgcatcaccgagtgggtcagcgtctgcaagtgagcggcccggggtcccctgggccctgatcgaggtcccctcctggagcctggccctctgaggcttacggtcttggcaaggcagcatcgccttggctcttgggaagaggagattggaccacatgatcttataggaacccctcagctctgaggtctgtgatcgccggacagtccaggcctgtctgaaccccaccactcacttctgtgggctctaggactgactgggttcttcctccctccccgaagcccagacagttcagttgggctgggggttgctccacaccctaaaacaagcctcagccaggcaacccgtcagtctgtctccatcctttcaccccttccctggagatgggaggtggggaatgaatggaagctgacgggcagagagaggaggattaaaaaaaagaaatagacataactgagctgaagtaattccataaagggcccagacagcctcctccaccattcccttcatcattcatttaacaaatatttattttgcactctctttgcggcactctgggggcggtggggtgcgtgggggtggcaatgcaaggcactgaggccacagatgtgagtaagcgagacacaacacttgtcctcttggaggttacattcttgctggggggaggcatgggcaataaacaagtaaatatacaaacaaaaaaaaaaaaaaaaaaa

SEQ ID NO: 18 - protein, 417 aa, Homo sapiens; identical to SEQ ID NO: 16

(19) United States
(12) Patent Application Publication
(10) Pub. No.: US 2014/0356333 A1
(43) Pub. Date: Dec. 4, 2014

(54) NAIL STEM CELLS AND METHODS OF USE THEREOF

(71) Applicants: Mayumi Ito, New York, NY (US); Makoto Takeo, Rego Park, NY (US)

(72) Inventors: Mayumi Ito, New York, NY (US); Makoto Takeo, Rego Park, NY (US)

(21) Appl. No.: 14/295,776

(22) Filed: Jun. 4, 2014

Related U.S. Application Data
(60) Provisional application No. 61/830,801, filed on Jun. 4, 2013.

(51) Int. Cl. C12N 5/0775; A61K 35/36

(52) U.S. Cl. CPC C12N 5/0668; A61K 35/36; C12N 2501/415
USPC 424/93.7; 435/377; 435/455

(57) ABSTRACT

The present invention is directed to an in vitro method for promoting proliferation, survival, and/or differentiation of K14+, K17+ nail stem cells (NSCs). The instant methods may be used to generate an expanded population of K14+, K17+ NSCs in vitro and expanded NSC populations in which a Wnt pathway is activated are envisioned as therapeutic agents. Methods for screening to identify agents capable of modulating K14+, K17+ NSC proliferation, survival, and/or differentiation are also encompassed herein, as are isolated, pure populations of homogeneous K14+, K17+ NSCs.

### CROSS REFERENCE TO RELATED APPLICATIONS

This application claims priority under 35 USC §119(e) from U.S. Provisional Application Ser. No. 61/830,801, filed Jun. 4, 2013, which application is herein specifically incorporated by reference in its entirety.

### GOVERNMENTAL SUPPORT

The research leading to the present invention was funded in part by NIH/NIAMS grant 1R01AR059768-01A1. The United States government has certain rights in the invention.

### FIELD OF THE INVENTION

The present invention pertains to the fields of multipotent cells and methods of generating and using same. More specifically, the invention relates to methods for isolating, expanding, and utilizing nail stem cells (NSCs), and methods for generating multipotent cells from NSCs. NSCs and multipotent cells generated therefrom can be used for regenerating amputated limbs and portions thereof, including digits, and regenerating/supplementing cell populations damaged by previous or ongoing disease in a subject or injury to a subject. Also encompassed are in vitro screening methods directed to identifying agents capable of promoting NSC expansion and functionality and modulating the generation of multipotent cells from NSCs. Homogenous populations of NSCs and multipotent cells generated therefrom made using the methods of the invention are also described and encompassed herein.

### BACKGROUND OF THE INVENTION

Several publications and patent documents are referenced in this application in order to more fully describe the state of the art to which this invention pertains. The disclosure of each of these publications and documents is incorporated by reference herein.

Amputations of limbs and smaller portions thereof, including distal tips of limbs and digits, can result from accidents in the work place or home, battlefield engagement, and/or surgical procedures designed to promote and preserve the life of the amputee. Fingertip amputations are, for example, one of the most common injuries presented in hospital emergency rooms. Clinical management strategies for fingertip amputations include, for example, reattachment of the severed tip when feasible, surgical intervention to promote tip reformation, which is generally associated with loss of digit length, and more conservative treatments that promote secondary healing. See, for example, Roshan et al. (J Hand Surg 37A:1287-1290, 2012). Each of these approaches can achieve a reasonable degree of success, but none typically achieves regeneration and restoration of the finger tip and normal functionality thereof or sensation therein.

In light of the challenges facing the field of regenerative medicine, developing strategies for human limb replacement that restore structure and function, while minimizing disfigurement, remain the major objective. Although spontaneous human fingertip regeneration has been documented in children, it is a rare event in adults. See, for example, Vidal et al. (J Hand Surg 18B:230-233, 1993). In contrast, salamanders are legendary for their ability to regenerate whole limbs following amputation and indeed use this property strategically when escaping from predators. Regeneration in mammals such as mice, however, is limited to digit tip regeneration (Rinkevich et al. Nature 476:409-413, 2011).

The citation of references herein shall not be construed as an admission that such is prior art to the present invention.

### SUMMARY OF INVENTION

In one aspect, the present invention is directed to a method for promoting tissue regeneration in a subject with an amputation that removed a body part, leaving a truncated body part attached to the subject, the method comprising administering to a distal tip of the truncated body part a cell population comprising nail stem cells contacted with at least one activator of a Wnt signaling pathway, wherein the administering promotes tissue regeneration in the subject, thereby restoring part or all of the amputated body part.

In an embodiment thereof, the body part amputated comprises one, two, or three digits of a finger or toe, a finger, a toe, or a limb or a portion thereof. In a more particular embodiment thereof, the amputated body part is a digit tip. As further described herein, the subject may be a mammal and, more particularly, may be a human.

In another embodiment, the cell population comprising nail stem cells is isolated from the proximal nail matrix. In a more particular embodiment, the cell population comprising nail stem cells comprises greater than 75%, 80%, 85%, 90%, 95%, 96%, 97%, 98%, or 99% K14+/K17+ double positive nail stem cells or comprises 100% K14+/K17+ double positive nail stem cells. The K14+/K17+ double positive nail stem cells may further be evaluated and/or selected on the basis of expressing high levels of Ki67 (Ki67^{high}).

In yet another embodiment, the cell population comprising nail stem cells is expanded in vitro prior to administering.

In a further embodiment, the cell population comprising nail stem cells comprises between 10³-10⁹ K14+/K17+ double positive nail stem cells.

In another embodiment, the cell population comprising nail stem cells consists of isolated proximal nail matrix cells. A combination of cellular markers that comprises K14+K17+ Gli2− is specific for the proximal nail matrix. In a more particular embodiment, the isolated proximal nail matrix cells exclude distal nail matrix cells. A combination of K14+K17−Gli2+ is specific for distal nail matrix cells. Accordingly, differential expression of K17 and Gli2 can be used to differentiate proximal from distal nail matrix cells.

In another embodiment, the cell population comprising nail stem cells is isolated from the body part amputated from the subject. In another embodiment, the cell population comprising nail stem cells is isolated from the subject or from an allogeneic subject.

As described herein and understood in the art, a number of activators of Wnt signaling pathways are known. Accordingly, the at least one activator of a Wnt signaling pathway may be any agent known to exhibit this property. Such agents may be selected, without limitation, from the following: Wnt3, Wnt 7a, Wnt7b, or Wnt10a. Nail stem cells may, moreover, be contacted with the at least one activator of a Wnt signaling pathway before, during, and/or after the administering. In an embodiment, the contacting is achieved by adding the at least one activator of a Wnt signaling pathway to the nail stem cells. In another embodiment, the contacting is achieved by expressing an exogenous activator of a Wnt signaling pathway in the nail stem cells. In a particular embodiment thereof, the nail stem cells are contacted with the at least one activator of a Wnt signaling pathway after in vitro expansion and immediately before administering/transplantation.

In another aspect, the present invention is directed to a method for promoting digit tip regeneration in a subject with an amputation that removed a digit tip leaving a truncated finger or toe attached to the subject, the method comprising administering to a distal tip of the truncated finger or toe a cell population comprising nail stem cells contacted with at least one activator of a Wnt signaling pathway. The subject may be a mammal and, more particularly, may be a human.

In an embodiment thereof, the cell population comprising nail stem cells is isolated from the proximal nail matrix.

In another embodiment, the cell population comprising nail stem cells is isolated from the proximal nail matrix. In a more particular embodiment, the cell population comprising nail stem cells comprises greater than 75%, 80%, 85%, 90%, 95%, 96%, 97%, 98%, or 99% K14+/K17+ double positive nail stem cells or comprises 100% K14+/K17+ double positive nail stem cells.

In yet another embodiment, the cell population comprising nail stem cells is expanded in vitro prior to administering. In a more particular embodiment, the cell population comprising nail stem cells comprises between 10³-10⁹ K14+/K17+ double positive nail stem cells.

In another embodiment, the cell population comprising nail stem cells consists of cells isolated from the proximal nail matrix. A combination of cellular markers that comprises K14+K17+ Gli2− is specific for the proximal nail matrix. In a more particular embodiment, the isolated proximal nail matrix cells exclude distal nail matrix cells. A combination of K14+K17−Gli2+ is specific for distal nail matrix. Accordingly, differential expression of K17 and Gli2 can be used to differentiate proximal from distal nail matrix cells.

In a particular embodiment, the cell population comprising nail stem cells is isolated from the digit tip amputated from the subject. The cell population comprising nail stem cells may also be isolated from a different digit tip of the subject or from an allogeneic subject. Allogeneic subjects suitable for this purpose include cadavers and fetuses.

The at least one activator of a Wnt signaling pathway may be any agent known to exhibit this property. Such agents may be selected, without limitation, from the following: Wnt3, Wnt7a, Wnt7b, or Wnt10a. Nail stem cells used for digit tip regeneration may be contacted/treated with the at least one activator of a Wnt signaling pathway before, during, and/or after the administering. In an embodiment, the contacting is achieved by adding the at least one activator of a Wnt signaling pathway to the nail stem cells. In another embodiment, the contacting is achieved by expressing an exogenous activator of a Wnt signaling pathway in the nail stem cells. In a particular embodiment, the nail stem cells are contacted with the at least one activator of a Wnt signaling pathway after in vitro expansion and immediately before transplantation.

Also encompassed herein is a method for expanding a population of nail stem cells in vitro and/or generating a population of cells having multipotent regenerative capacity from isolated nail stem cells in vitro, the method comprising isolating tissue comprising nail stem cells from a subject, culturing the nail stem cells to expand the population of nail stem cells in vitro, and activating Wnt signaling in the expanded population of nail stem cells, wherein the culturing induces proliferation of the nail stem cells and the activating promotes differentiative capacity of the nail stem cells, thereby expanding the population of nail stem cells and/or generating the population of cells having multipotent regenerative capacity.

With regard to sources of nail stem cells, approximately 1×10⁴ nail stem cells can be isolated from a mouse nail matrix, approximately 1×10⁵ nail stem cells can be isolated from a rat nail matrix, and approximately 1×10⁶ nail stem cells can be isolated from a human nail matrix.

In an embodiment thereof, the tissue comprising nail stem cells is nail matrix. More particularly, the nail matrix is proximal nail matrix. Proximal nail matrix is characterized as K14+K17+ Gli2−.

In a particular embodiment, the activating is achieved by contacting the nail stem cells with at least one activator of a Wnt signaling pathway. Such activators include, without limitation, Wnt3, Wnt7a, Wnt7b, or Wnt10a. In another embodiment, the activating is achieved by transfecting or transducing the nail stem cells with a vector comprising a nucleic acid sequence encoding an exogenous polypeptide that activates a Wnt signaling pathway in transduced nail stem cells. As described herein, the exogenous polypeptide that activates a Wnt signaling pathway may encode a Wnt ligand, such as Wnt3, Wnt7a, Wnt7b, or Wnt10a. A non-limiting range of 0.1-100 μM Wnt activator/ligand is envisioned for use in methods described herein, either with respect to the amount of Wnt ligand with which the nail stem cells are contacted or the amount of Wnt ligand expressed following transduction. With the respect to timing, in a particular embodiment, activating the Wnt pathway takes place after expanding the nail stem cell population in vitro and immediately before administering/transplanting into the subject in need thereof. As further described herein, an exogenous polypeptide that activates Wnt signaling pathway may encode a stabilized form of β-catenin.

In an embodiment, the subject from whence the tissue comprising the nail stem cells is isolated is a mammal. In a more particular embodiment, the tissue is isolated from a human.

As described herein, Wnt-activated NSCs give rise to keratinized nail cells, nail bed and nail plate. In association with the formation of these structures, NSC derived cells emit signals that attract nerves required for initiating FGF2 signaling that promotes underlying mesenchymal cell regeneration.

The method for expanding a population of nail stem cells in vitro and/or generating a population of cells having multipotent regenerative capacity from isolated nail stem cells in vitro may further comprise administering the expanded population of nail stem cells or the population of cells having multipotent regenerative capacity to a recipient in need thereof. Such recipients in need thereof may be afflicted with, for example, nail dystrophy or nail psoriasis. In circumstances in which the donor (subject from whom the tissue comprising the nail stem cells is isolated) and the recipient are the same individual, the nail stem cell transplant may be referred to as an autologous transplant. In circumstances wherein the donor and recipient are different individuals of the same species, the nail stem cell transplant may be referred to as an allogeneic transplant.

Also encompassed herein is an isolated, homogeneous population of K14+/K17+ double positive nail stem cells, wherein the isolated, homogeneous population comprises about or at least between 10³-10⁹ K14+/K17+ double positive nail stem cells, wherein a Wnt signaling pathway is activated. Activation of Wnt signaling pathways leads to expression of TCF1 in the isolated, homogeneous population K14+/K17+ double positive nail stem cells. Expression of TCF1 is, therefore, both a marker of Wnt activation and a distinguishing feature of the isolated, homogenous population of K14+/K17+ double positive nail stem cells. Yet another distinguishing feature of the isolated, homogenous population of K14+/K17+ double positive nail stem cells described herein is the absence of Gli2 expression.

In a particular embodiment, the Wnt signaling pathway in the isolated, homogeneous population of K14+/K17+ double positive nail stem cells is activated by contacting the K14+/K17+ double positive nail stem cells with a polypeptide that activates the Wnt signaling pathway. Exemplary such polypeptides include, without limitation, Wnt3, Wnt7a, Wnt7b, and Wnt10a.

In another particular embodiment, the Wnt signaling pathway in the isolated, homogeneous population of K14+/K17+ double positive nail stem cells is activated via expression of an exogenous polypeptide or shRNA/siRNA that activates a Wnt signaling pathway in the K14+/K17+ double positive nail stem cells. Further to the above, the isolated, homogeneous population of K14+/K17+ double positive nail stem cells may comprise an exogenous polypeptide or agent (e.g., an sh/RNA/siRNA that inhibits a negative regulator of the Wnt signaling pathway) that activates a Wnt signaling pathway in the K14+/K17+ double positive nail stem cells. Exemplary such exogenous polypeptides include, without limitation, Wnt3, Wnt7a, Wnt7b, and Wnt10a.

In yet another embodiment, the Wnt signaling pathway is constitutively activated in the isolated, homogeneous population of K14+/K17+ double positive nail stem cells via expression of an exogenous polypeptide that constitutively activates a Wnt signaling pathway in the K14+/K17+ double positive nail stem cells. More particularly, the exogenous polypeptide that activates Wnt signaling pathway encodes a stabilized form of β-catenin.

In a further aspect, a method for screening to identify an agent that modulates nail stem cell proliferation in vitro is presented, the method comprising the steps of: a) isolating a population of K14⁺ nail stem cells from a mammal and dividing the population into at least a first and second sub-population of K14⁺ nail stem cells; b) co-culturing a first sub-population of K14⁺ nail stem cells with a fibroblast feeder layer in media supplemented with serum, wherein the co-culturing promotes proliferation of the K14+ nail stem cells; c) co-culturing a second sub-population of K14⁺ nail stem cells with a fibroblast feeder layer in media supplemented with serum, and an agent; and d) comparing the number of K14+ nail stem cells in each of said first and second sub-populations of K14+ nail stem cells incubated without or with the agent, wherein a change in number of K14+ nail stem cells following incubation with the agent relative to the number of K14+ nail stem cells following incubation without the agent indicates that the agent is a modulator of K14+ nail stem cell proliferation in vitro. Agents suitable for evaluation in the aforementioned screening method include, without limitation, small molecules; polypeptides; shRNA/siRNA; and neutralizing/blocking antibodies. The method may further comprise co-culturing the first and second sub-populations of K14⁺ nail stem cells with an activator of a Wnt signaling pathway. In a further embodiment, the method comprises assessing K17+ expression in the first and second sub-populations of K14⁺ nail stem cells.

Other features and advantages of the invention will be apparent from the following description of the particular embodiments thereof, and from the claims.

### BRIEF DESCRIPTION OF THE DRAWINGS

FIG. 1. NSCs are harbored in the proximal nail matrix. a. Experimental scheme. B, c. Whole mount (b) and sectioned (c) specimens of K14-CreER;R26R reporter mice. LacZ expression was detected at indicated times after TAM treatment. Inset in (b) shows top view of the nail. d, Quantitative analysis of LacZ positive streaks. e, Tissue section analysis of a LacZ⁺ colony at 5 mo after chase and schematic representation, to illustrate cell lineages from proximal matrix cells. f. A typical nail sample used for microdissection to obtain proximal, distal and bed fragments. g, Immunocytochemistry for K14 and K17 using single cell suspensions from each compartment. h-j, In vitro colony forming assay with single cell suspensions obtained from indicated fragments. Visualization of colonies by Rhodamine B staining (h) and quantification of colonies>3 mm² (i). Brightfield images of proximal and distal nail epithelial colonies (j). Arrowheads indicate LacZ⁺ cell or colony. Dashed lines delineate the boundary between nail epithelium and underlying connective tissue (ct). Asterisk indicates nonspecific background. Data are presented as the mean±SD. Scale bars, 500 μm in (b and f); 100 μm in (c and e). dm, distal matrix, kz, keratogenous zone, nb, nail bed, np, nail plate, pm, proximal matrix.

FIG. 2. Epithelial β-catenin is required for nail differentiation. a. Experimental scheme. Three-week-old K14-CreER;β-catenin cKO mice and littermates were treated with TAM for 7 d, and analyzed at 2 mo after TAM treatment. b-e. Gross appearance (b and d) and H&E staining (c and e) of control (b and c) and cKO digit (d and e). f-h. Immunofluorescence for indicated markers at 2 mo after TAM treatment. i. Summary of immunohistochemistry in (f-h). Dashed lines indicate the border between nail basal layer and connective tissue. Lines indicate the outline of nail plate in (f-h). Asterisks show nonspecific background. Scale bars, 500 μm in b, c, f.

FIG. 3. Nail epithelial β-catenin is required for blastema growth and digit regeneration. a. Experimental scheme. Three-week-old old K14-CreER;β-catenin cKO mice and littermates were treated with TAM for 7 d immediately after distal tip amputation, and analyzed at the indicated time points. b, Gross appearance of regenerated digit at 5 w after amputation. c. Whole mount alizarin red analysis. d. Trichrome staining. e and f. Quantification analyses of the nail length (h) and the bone length (i) at 5 w after amputation. g. Analysis of Wnt activation in regenerating nail epithelium using TOPGAL at 3 w after amputation. Lower panel is the schematic illustration of the upper panel. h. Quantitative analyses of the distance between nerve tip and wound epidermis and the innervations at 3w after amputation. i. Proliferation analyses by Ki67 immunohystochemistry at 3w after amputation. Red bar in o indicates the average. Dashed lines, border between nail epithelium and connective tissue. Arrows Asterisks in (m, o and r) indicate autofluorescence from blood cells. Data are presented as the mean±SD. Scale bars, 500 μm in (b-d); 100 μm in (h).

FIG. 4. Forced Wnt activation in wound epidermis can overcome the limitation of regeneration following proximal amputation. a. Experimental scheme. Three-week-old K14-CreER;β-catenin^{fl/ex3} (mutant) mice and littermate controls were treated with TAM for 7 days starting from 2 w after amputation at the proximal level. b-f, Immunohistochemical analyses with indicated markers at 3 w after amputation. g, Gross appearance of regenerated digits. h, Whole mount alizarin red analysis. i and j, Quantification analyses of the nail (i) and bone length (j) at 4 w after amputation. Red bars in n show the meal values. Arrows in (c) and (e) indicate Tcf1⁻ proximal matrix and FGF2⁺ epidermis, respectively. Arrowheads in d point nerves. Dotted lines in b, c, g and h indicates amputation plane. Dashed lines indicate the border between epidermis and connective tissue. Dotted lines in (b) indicate amputation planes. Quantified data are presented as the mean±SD. Scale bar, 100 μm in (b-f), 500 μm in (g and h).

FIG. S1. Proposed model: Wnt activation in the nail epithelium provides a molecular link between nail and digit regeneration. Under homeostatic conditions, NSCs give rise to distal matrix cells with Wnt activation. Simultaneously, NSCs and distal matrix cells differentiate into the nail plate. After distal level amputation, the wound site is covered by regenerating nail epithelial cells, which in turn activate Wnt signaling to differentiate into distal matrix cells and the nail plate. In addition, this Wnt activation promotes blastema innervations, which is necessary for Fgf2 expression in the regenerating nail epithelium. FGF2 then promotes proliferation of Runx2 positive mesenchymal cells, ultimately leading to digit regeneration. In contrast, digit amputation at the proximal level results in the depletion of Wls expressing nail epithelium, resulting in the absence of Wnt activation in the nail epithelium and the failure to regenerate the nail and digit.

FIG. S2. Digit regeneration is associated with nail regeneration. a and b. Experimental scheme. Digits of 3-week-old wild type mice were amputated at proximal (a) and distal levels (b), and analyzed at 5 w after amputation. c and d. Whole mount alcian blue/arizarin red staining at 5 w after proximal (c) or distal (d) amputation. e, Quantitative analysis of bone length at 5 w after amputation. Note that neither nail nor digit regeneration occurs after proximal amputation. Data are presented as the mean±SD.

FIG. S3. Proximal nail matrix cells are biochemically and morphologically distinct from distal matrix cells. a-c, Immunohistochemistry for indicated markers identified two biochemically distinct compartments within the nail matrix: proximal matrix cells (K14+K17+ Ki67high), and distal matrix cells (K14+K17−Ki67low). Nail bed, adjacent to the distal matrix, does not proliferate (K14+K17−Ki67−). d, Ultrastructural analysis by TEM. Proximal nail matrix cells exhibit morphological characteristics of undifferentiated cells (white arrow), including less developed organelles and fewer intra-digitations between the cells, compared to distal nail matrix and bed cells (black arrows). Nuclei of proximal nail matrix possess less condensed chromatin, indicative of actively proliferating cells. e. Hematoxylin and eosin staining. f. Schematic illustration of digit anatomy and marker expression. Dashed lines, border between nail basal layer and connective tissue. kz, keratogenous zone. Scale bars, 100 μm in (a); 10 μm in (d).

FIG. S4. Lineage analysis of nail epithelium including the nail fold. a-c. Three-week-old K14-CreER; R26R mice were treated once with TAM, resulting in sporadic genetic labeling K14+ nail epithelial cells with LacZ. Intact nails, with the untrimmed nail fold epithelium enveloping the nail plate, were incubated with X-gal. The nail fold overlying the proximal part of nail was carefully flipped up to trace the lineage in the fold and matrix (a). Progeny of labeled cells began extending distally by 7 d after TAM (b) and produced LacZ+ colonies within nail tissues by 1 mo after TAM (c). Note that LacZ-labeled cells were found within the nail fold but they were not associated with lineage of the nail from the matrix (right panel). Right panels are higher magnifications of the left panels. Dashed lines indicate the border between the nail plate and nail fold. Scale bars, 500 μm in left panel; 100 μm in right panel.

FIG. S5. Wnt signaling is suppressed in NSCs. a and b. X-gal staining on cryosections of nails from TOPGAL (a) and Axin2-LacZ (b) reporter mice. In Topgal mice, LacZ expression is mainly observed in keratogenous zone and occasionally found in basal cells in the proximal matrix as previously reported. LacZ signal is absent in the proximal matrix (a). LacZ signal in Axin2-LacZ mice was found more proximally in nail matrix than Topgal mice. Insets indicate immunohistochemistry for K17 on LacZ stained sections, showing that the distal part of K17 expressing proximal matrix area overlaps with Axin2-LacZ expression. c and d. Immunohistochemistry for Tcf1 (c) and Wls (d). Consistent with reporter mice, Tcf1, a Wnt signaling mediator, was strongly expressed in distal matrix (c). The expression of Wls gene which is required for Wnt ligand(s) secretion is detected only in Wnt activated nail epithelial cells (arrowheads in d). Dashed lines indicate the border between nail epithelium and connective tissue. Scale bars, 100 μm.

FIG. S6. Epithelial Wls is required for nail differentiation. Three-week-old K14-CreER;Wls cKO mice and littermates were treated with TAM for 7 d, and analyzed at 2 mo after TAM treatment. a-d. Gross appearance (a and c) and H&E staining (b and d) of control (a and b) and cKO digit (c and d). Failure to maintain nail growth becomes grossly apparent in cKO mice (a and c). H&E stained sections (b and d) showed the failure to form the keratogenous nail plate. e-g, Immunofluorescence for indicated markers at 2 mo after TAM treatment. Dashed lines indicate the border between nail basal layer and connective tissue. Lines indicate the outline of nail plate in e-g. Scale bars, 500 μm in (a and b); 500 μm in (e).

FIG. S7. Reepithelialization occurs normally without epithelial β-catenin.

Three-week-old K14-CreER;β-cateninfl/fl (cKO) mice and control mice were treated with TAM for 7 days immediately after amputation at the distal level. Digits were harvested at indicated time points and were subjected to either trichrome staining (a, c, e, g, i and k) or immunofluorescence for β-catenin (b, d, f, h, j and l), and the percentage of re-epithelialized digit (m) and the percentage of nuclear β-catenin positive cells (n) was analyzed. 1 week after amputation, re-epithelialization was not yet complete in both control and cKO (a, g). By 2 weeks after amputation, both control and cKO mice completed re-epithelialization (c, i and m). In control mice, nuclear β-catenin is observed 3 w after amputation (f, n), but absent during (b) and immediately after (d) re-epithelialization. In cKO mice, β-catenin immunoreactivity was not detected in the wound epidermis throughout regeneration (h, j, l and n). Brackets in a and g indicate the gap in epithelium. Dashed lines indicate border between basal epidermis and connective tissue. Data are presented as the mean±SD. Scale bars, 500 μm in (a, c and g); 10 μm in (b).

FIG. S8. Failure in proliferation of Runx2+ cells and Sp7+ cells, and BMP4 expression upon loss of epithelial β-catenin. a. Experimental scheme. Three-week-old K14-CreER;β-catenin cKO mice and littermates were treated with TAM for 7 d immediately after distal tip amputation, and analyzed at 3 w after amputation. a-g. Immunohystochemistry (b, c, e, f) or in situ hybridization (d, g) for indicated markers. Insets indicate high magnification image. f. Quantification analyses of the percentage of Runx2 or Sp7 positive cells among PCNA positive cells in wild type digits at 3 w after amputation. g. the percentage of PCNA positive cells in Runx2 or Sp7 positive cells underneath the wound epidermis at 3 w after amputation. h. Dashed lines, border between nail epithelium and connective tissue. Arrowheads indicate Runx2/PCNA or Sp7/PCNA double positive cells. Asterisks indicate autofluorescence from blood cells. Data are presented as the mean±SD. Scale bar, 100 μm.

FIG. S9. Spatial association between nerves and Runx2 or Sp7 positive mesenchymal cells. Digits of 3-week-old wild type mice were amputated at the distal level and harvested at 3 w after amputation. a and b. Double immunohistochemistry for PCNA with mesenchymal cell marker Runx2 (a) or SP7 (b). c. Schematic illustration showing the distribution of nerves and mesencymal cells. Dashed lines indicate the border between nail epithelium and connective tissue. Arrowheads indicate nerve. Asterisks indicate autofluorescence from blood cells. Scale bar, 100 μm.

FIG. S10. Sema 5a is upregulated in control nail epithelium at 3 w after amputation. a-c. Three-week-old K14-CreER;β-cateninfl/fl (cKO) mice and control mice were treated with TAM for 7 days immediately after amputation at the distal level. Digits were harvested at indicated time points and subjected to qPCR for Sema5a. Experimental scheme and quantification analysis of Sema5a expression in regenerating digit tip including nail epithelium and mesenchymal cells in control and cKO mice at 3 w after amputation (a), in control digit tip at 2 w and 3 w after amputation (b), and in control nail epithelium and mesenchymal cells at 3 w after amputation (c). Data are presented as the mean±SD.

FIG. S11. Denervation inhibits blastema growth. a. Experimental scheme. Denervation was performed by removing a 2-3 mm segment of the sciatic nerve from the right hind limb of 2-week-old mice. Digits were amputated at 3-weeks-old, and analyzed at indicated time points. Digits of left hind limb were used as an internal control. b and e. Immunohistochemistry for Ki67 at 3 w after amputation. c and f, Gross appearance of regenerated digit. d and g, Whole mount alizarin red analysis. h and i, Quantification analyses of the nail (h) and bone length (i) at 5 w after amputation. Dashed lines indicate the border between nail basal layer and connective tissue. Data are presented as the mean±SD. Scale bars, 500 μm.

FIG. S12. Innervation is required for Fgf2 and phospho-ERK expression in regenerating nail epithelium and underlying mesencymal cells. a-l. Denervation was performed on 2-week-old mice. Digits were amputated at 3-week-old, and analyzed at indicated time points. Immunohistochemistry for indicated markers at 2 w and 3 w after amputation on control (a, b, e, f and i), denervated (c and g) and K14-CreER;β-catenin cKO digit (d and h). Expression analysis of FGF receptors by real-time qPCR at 3 w after amputation (j). Quantification analyses of the percentage of FGF2+ cells in wound epidermis or underlying mesenchymal cells (k) and the percentage of Runx2 positive cells in pERK positive cells (l). Inset indicates high magnification view of boxed area. Dashed lines, border between nail epithelium and connective tissue. Arrows in b indicates FGF2 expressing cells. Arrowheads in f and i indicate pERK and Runx2/pERK expressing cells, respectively. Data are presented as the mean±SD. Scale bars, 500 μm in (a); 100 μm in (e).

FIG. S13. Wnt ligand secretion from epithelial cells is required for blastema growth and digit regeneration. a. Experimental scheme. Three-week-old K14-CreER; wntless (Wls) cKO mice and littermates were treated with TAM for 7 d immediately after distal tip amputation, and analyzed at the indicated time points. b-m. Immunohistochemistry for indicated markers on control (b-g) and Wls cKO digit (h-m). In Wls cKO mice, Tcf1 and FGF2 expression was absent in regenerating distal matrix cells (c, i), and fewer innervations (d, j) and a reduced number of Ki67 positive cells (g, m) were observed underneath the nail epithelium. n-p. Quantitative analyses. q. Gross appearance of regenerated digit. r. Whole mount alizarin red staining at 5 w after amputation. s and t. Quantification of the nail length (s) and the bone length (t) at 5 w after amputation. Dashed lines indicate the border between nail epithelium and connective tissue. Arrows and arrowheads indicate pERK expressing cells. Asterisks in h indicate autofluorescence from blood cells. Data are presented as the mean±SD. Scale bars, 100 μm in (b); 500 μm in (q).

FIG. S14. FGF2 promotes mesenchymal cell proliferation in vitro and in vivo. a-i, in vitro blastema cell proliferation assay. Mesenchymal cells were isolated from blastema at 3 w after amputation and cultured with (b-d) or without (a) recombinant Fgf2 protein and cell proliferation was analyzed at 2 w after culture by immunocytochemistry for the proliferation marker, Ki67, and quantified (e and f). Alizarin red staining of blastema cells after 3 w in culture in bone differentiation media (g-i). j-o. Denervation was performed on 2-week-old mice and digits were amputated in 3-week-old mice. Two weeks after amputation when re-epithelialization was complete, FGF2-soaked beads were implanted into the amputation site (j). Digits were harvested at 3 w after amputation. Immunohistochemistry for indicated markers 3 w after amputation (k, l, m and n). Quantitative analysis of the percentage of PCNA-positive cells in Runx2-positive cells at 3 w after amputation (o). Arrowheads indicate pERK and PCNA/Runx2 positive cells in n and o, respectively. Insets in n indicate high magnification image. Asterisks indicate beads. Dashed lines indicate the border between epidermis and connective tissue. Data are presented as the mean±SD. Scale bar, 100 μm in (k).

FIG. S15. Proximal amputation removes entire Wls expressing, but not K17 positive nail epithelium. a and b. Experimental scheme. Digits of 3-week-old Axin2-LacZ mice were amputated at proximal and distal level, and amputated digits were subjected to further analysis. c-f. Immunohistochemistry for indicated markers. Following proximal amputation, K17+ NSCs niche is still observed (c). In contrast, Wls expression is not detected in the remaining part after proximal amputation (e) unlike after distal amputation (f). Dashed lines indicate the border between nail basal layer and connective tissue. Arrowheads indicate Wls positive area. Scale bar, 100 μm.

FIG. S16. β-catenin stabilization in the epithelium promotes proliferation of Runx2 positive mesenchymal cells. a. Experimental scheme. Three-week-old K14-CreER;β-cateninfl/ex3 (mutant) mice and littermate controls were treated with TAM for 7 d starting from 2 w after amputation at the proximal level. b-e, Immunohistochemistry for indicated markers at 3 w after amputation. f. Quantification analysis of the percentage of Runx2 or Sp7 positive cells in PCNA positive cells underneath the wound epidermis at 3 w after amputation. The proliferation of Runx2+ mesenchymal cell is significantly upregulated following β-catenin stabilization in the epithelium. Arrowheads indicate Runx2/PCNA or Sp7/PCNA double positive cells. Insets indicate high magnification view. Asterisks indicate autofluorescence from blood cells. Data are presented as the mean±SD. Scale bar, 100 μm.

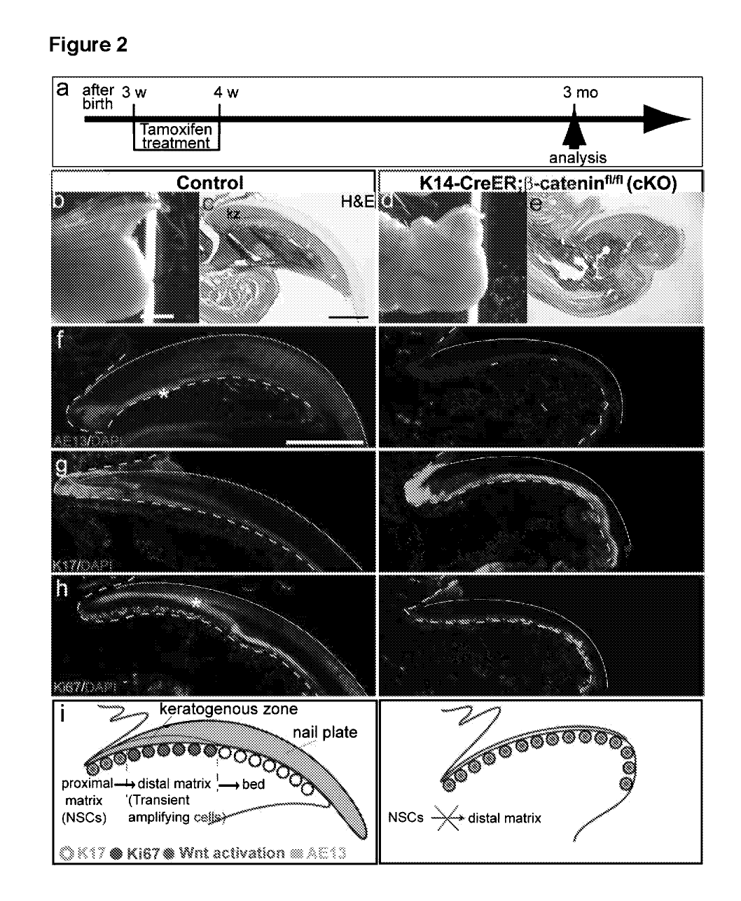

FIG. S17. β-catenin stabilization in epithelium does not affect nail differentiation in homeostasis. a-f. Three-week-old K14-CreER;β-catenin^{fl/ex3} (mutant) mice and littermate controls were treated with TAM for 7 days, and analyzed at 2 w after TAM treatment. Gross appearance of control (a) and mutant (b). Both control and mutant had well developed nail plate. Immunofluorescence for indicated markers (c-f). Note the expression pattern is similar to wild type (Supplementary FIG. 2). Insets indicate high magnification view of boxed area. Dashed lines indicate the border between nail basal layer and connective tissue. Scale bars, 500 μm in (a); 100 μm in (c).

FIG. S18. β-catenin stabilization in the epithelium does not promote digit regeneration after amputation proximal to the NSC niche. a. Experimental scheme. 3-week-old K14-CreER;β-catenin^{fl/ex3} (mutant) mice and littermate controls were treated with TAM for 7 d starting from 2 w after amputation proximal to the NSC niche. b, c, e and f. Immunohistochemistry for indicated markers at 3 w after amputation. d and g. Whole mount alizarin red staining on control (d) and mutant digit (g) at 5 w after amputation proximal to the NSCs niche. h. Quantification of the bone length at 5 w after amputation. i-k. Detection of PCNA with Runx2 (i) and Sp7 (j) shows mesenchymal cells with bone differentiation potential are present but are not induced to proliferate (k). Dashed lines indicate the border between epidermis and connective tissue. Data are presented as the mean±SD. Scale bar, 100 μm in (b); 500 μm in (d and i).

### DETAILED DESCRIPTION OF THE INVENTION

More than 1.7 million people with amputations are living in the U.S. This number increases each year, with over 113,000 people requiring operations for limb amputations due to injuries and disease. Although major investment has been made in prosthetic technology to aid amputees, no prosthetics have been devised that are comparable to actual limbs and thus, amputees continue to face many limitations in everyday life even with the best prosthetic equipment. Currently there is no therapy to promote the regeneration of amputated limbs or digits. Understanding the biology of tissue/organ regeneration is, therefore, necessary to develop a cellular and/or pharmacological therapy to promote regeneration and results presented herein advance our understanding the biology of regeneration.

As described herein above, certain animals such as newts possess a high capacity for regeneration (Kumar et al., 2007) and can faithfully regenerate lost parts of a limb following amputation (Ito et al., 1999). By contrast, mammals typically fail to regenerate tissue/organs to its original collective form following tissue loss by injury. Nevertheless, previous studies revealed that adult mice can regenerate the entire hair follicle, a mini complex organ in the skin, after wounding (Ito et al., 2007). Moreover, the digit tip that includes the terminal phalanx (distal-most bone of the limb) can regenerate upon amputation in humans and mice (Douglas, 1972, Illingworth, 1974, Borgens 1982, Neufeld, and Zhao, 1995). These observations represent rare and understudied, but actual examples of epimorphic regeneration in mammals that mirror amphibian limb regeneration.

Digit tip regeneration involves the coordinated re-growth of the nail organ, which includes several distinct types of nail epithelial cells and the digit bone (Han et al., 2008). Upon amputation of the digit tip, epithelial cells migrate to re-epithelialize the wound surface followed by accumulation of undifferentiated mesenchymal cells, referred to as the blastema, in the amputated area (Neufeld et al., 1980, Fernando et al., 2011). Although the cellular origins of the blastema have not been completely defined, recent studies have demonstrated that these cells originate from cells of mesodermal origin, rather than from cells of ectodermal origin (Rinkevich et al., 2011, Lehoczky et al., 2011). The present inventors have, furthermore, found that the majority of blastema cells express Runx2, a marker for osteo-lineage commitment (Ducy et al., 1997).

Epithelial signals have been shown to be essential for the formation and growth of mesenchymal blastema in amphibian limb regeneration. Removal of the wound epidermis covering the amputation site precludes regeneration of the underlying mesenchyme (Goss 1956, Thornton 1957 and Mescher 1976). It is noteworthy that signals derived from nerves also mediate the interactions (Brockes 1987), which finding underscores the collaborative nature of the regenerative process whereby multiple cell types orchestrate the regeneration of complex organs similar to embryonic organogenesis (Zhang et al., 2008). Several paracrine signals, including Wnt signaling and FGF signaling have been implicated in the regulation of limb regeneration (Mullen et al., 1996, Kawakami et al., 2006, Yokoyama et al., 2007). Studies in lower vertebrates are limited, however, by challenges associated with targeting specific cell populations in these tissues. Moreover, the mechanisms identified in lower vertebrates appear to differ substantially from those underlying mammalian regeneration.

As indicated herein, mammalian digit-tip can regenerate upon amputation^{1,2}, like amphibians. It is unknown why this capacity is limited to the area associated with the nail²⁻⁴. The present inventors demonstrate herein that nail stem cells (NSCs) reside in the proximal nail matrix and that the mechanisms governing NSC differentiation are directly coupled with their ability to orchestrate digit regeneration. Early nail progenitors undergo Wnt-dependent differentiation into the nail. Upon amputation, this Wnt activation is required for nail regeneration and also for attracting nerves that promote mesenchymal blastema growth, leading to the regeneration of the entire digit. Amputations proximal to the Wnt-active nail progenitors result in the failure to regenerate the nail/digit (Supplementary FIG. S1). Nevertheless, β-catenin stabilization in the NSC region induced their regeneration. These results establish a link between NSC differentiation and digit regeneration, suggesting a utility of the NSCs in developing novel treatments for amputees.

Digit tip regeneration seen in both mice and humans involves the coordinated re-growth of the nail organ, including nail epithelial cells, and the terminal phalanx. Upon regrowth of the nail after amputation of the digit tip, undifferentiated mesenchymal cells including fate-restricted progenitor cells^{5,6} accumulate under the wound epithelium and form the so-called blastema⁷. Growth and differentiation of these mesenchymal cells leads to digit regeneration. However, neither nail nor digit regenerates when amputated proximally to the nail^{2-4,8,9} (Supplementary FIG. S2) and it is unknown why this limitation exists. Previous studies showed that nail transplantation following proximal digit amputation can induce ectopic digit bone differentiation⁴, leading to a hypothesis that the nail epithelium has a special function in digit regeneration. Testing this hypothesis may provide an understanding of why regeneration is limited to the nail-associated part of digits, and how epithelial cells can influence underlying mesenchymal cells to regenerate digit bone. The role of the nail epithelium in digit regeneration has remained elusive, however, due in part to the lack of lineage and molecular analyses of normal nail epithelium.

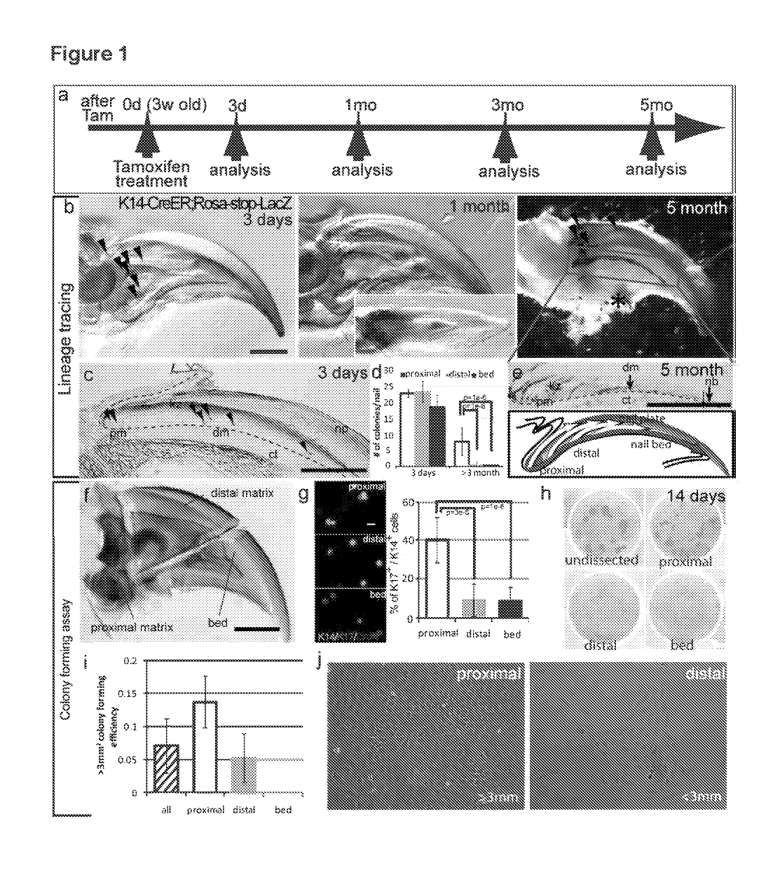

To locate NSCs, the present inventors performed lineage tracing using K14-creER; R26R-lacZ reporter mice. A single injection of tamoxifen (TAM) genetically labeled a small subset of K14⁺ nail basal epidermal cells, including nail matrix cells and bed cells, with LacZ (FIG. 1b, c). Over time, descendants of the labeled K14⁺ nail epithelial cells extended linearly and distally, reflecting the direction of their growth (FIG. 1b). By 3 months after labeling, the number of LacZ⁺ streaks emanating from the distal part of matrix and the bed decreased significantly (FIG. 1d). In contrast, the streaks emerging from the proximal matrix persisted for at least 5 months (FIG. 1b, d). These streaks included the proximal matrix, distal matrix and bed cells (FIG. 1e). The progeny of proximal matrix and distal matrix both migrated vertically to give rise to individual keratinized layers of the nail plate¹⁰. These results show that the proximal matrix contains self-renewing NSCs that sustain nail growth. LacZ⁺ colonies in the nail fold, the epithelium surrounding the nail, were discontinuous from the streaks that gave rise to the nail plate, suggesting that the nail fold did not contribute to the cells for nail growth (Supplementary FIG. S4).

Histological analyses revealed that proximal matrix cells possessed less interdigitations, characteristic of undifferentiated epidermal cells (Supplementary FIG. S3). Immunohistochemistry with proliferation and epidermal differentiation markers¹¹ found that proximal matrix cells containing NSCs were highly proliferative (Ki67^{high}) and expressed K17 in addition to K14 (Supplementary FIG. S3). Isolated proximal matrix cells, enriched with K14⁺K17⁺ expression (FIG. 1f, g), exhibited the highest colony forming ability in vitro, a general characteristic of epithelial stem cells (FIG. 1h-j).

To understand molecular mechanisms underlying NSC differentiation, the present inventors generated a microarray of proximal matrix versus distal matrix. Most strikingly, the analyses revealed that proximal matrix cells enriched with NSCs downregulated Wnt signaling pathway genes, which is known to regulate embryonic development of limb/nail organ¹²⁻¹⁴ as well as differentiation of epithelial and melanocyte stem cells¹⁵. Analyses with Wnt reporter mice showed that Axin2-LacZ signal started from the distal part of the K17⁺ NSC region and persisted into the distal matrix, whereas the TOPGAL signal was seen in the K17⁺ distal matrix^{17,18}. Although these two markers distribute differently¹⁶, both signals were absent in the proximal end of the nail matrix (Supplementary FIG. S5). Additionally, TCF1, a nuclear mediator of Wnt signaling¹⁹, and Wntless (Wls), required for Wnt ligand secretion²⁰, were missing in the proximal end of the matrix. Moreover, several keratins that contained a Tcf-1/Lef-1 consensus binding site were upregulated in the distal matrix compared with NSC region (Supplementary Table 1)^{21,22}, suggesting direct involvement of Wnt signaling in nail differentiation.

Supplementary Table 1. List of keratin
and keratin associated genes that are
downregulated in proximal matrix cells

| Gene | fold | Lef/Tcfl binding sites in |
|---|---|---|
| name | change | putative promoter region |
| Krtcap2 | -1.1 | ATCAAAG |
| Krt36 | -1.4 | AACAAAG, ATCAAAG, |
| | | TACAAAG, ATCAAAG, |
| | | TTCAAAG |
| Ktr32 | -1.5 | TACAAAG, TTCAAAG, |
| | | TACAAAG |
| Kttcap3 | -4.0 | AACAAAG |
| Krtap16-7 | -4.6 | TACAAAG, ATCAAAG |
| Krtap9-3 | -5.2 | ATCAAAG |
| Krtap6-1 | -5.4 | TTCAAAG |
| Krtap28-13 | -27.1 | TTCAAAG |

To verify the role of Wnt activation in the nail epithelium, the present inventors deleted β-catenin, an essential mediator of Wnt signaling, in adult epithelium using K14-CreER;β-catenin^{fl/fl} (cKO) mice (FIG. 2a). By two months after induction of β-catenin deletion by TAM treatment, nail formation is abrogated (FIG. 2b-e), as revealed by the lack of AE13, a marker for keratinized nail cells²² (FIG. 2f). Moreover, the entire nail epithelium showed characteristics of the NSC region (K17⁺Ki67^{high}) (FIG. 2g-i). Similar defects were observed in another mouse model (K14-CreER;Wntless^{fl/fl}) that depletes Wls in epithelial cells, confirming the essential role for Wnt signaling in nail differentiation (Supplementary FIG. S6).

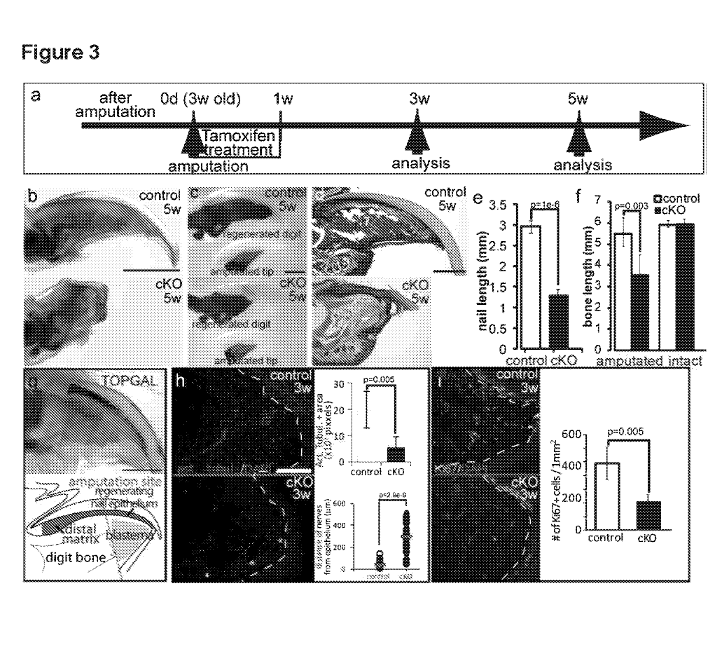

Next, to determine how nail differentiation is linked to digit regeneration upon amputation, the present inventors treated cKO mice with TAM beginning immediately after digit amputation (FIG. 3a). The present inventors focused on digit bone regeneration to evaluate the completeness of the regenerative response since muscle and tendon are absent at this amputation level⁶. In control mice, the nail resumed its original structure by 5 weeks after amputation (FIG. 3b), and the amputated digit bone regenerated along with nail regeneration (FIG. 3c-f). In cKO mice, the nail failed to regenerate as expected due to the essential role for Wnt signaling in nail differentiation (FIG. 3b, e). Remarkably, bone regeneration in these mice was also completely blocked (FIG. 3c, d, f). Intact non-amputated digits in cKO mice (internal control) maintained similar digit bone length compared with intact digits in control mice at 5 weeks after TAM treatment (FIG. 3f).

Time-course studies showed that β-catenin was clearly depleted in nail epithelial cells of cKO mice by one week after TAM induction (Supplementary FIG. S7). Nonetheless, the amputated areas of both control and cKO mice were similarly re-epithelialized two weeks after amputation. In control mice, the regenerating nail matrix displayed Wnt activation with TOPGAL activity (FIG. 3g), contiguous with the original nail matrix cells, which permitted nail differentiation. Underneath the Wnt-active regenerating matrix, mesenchymal cells were actively proliferating (FIG. 3i). The majority (about 90%) of these proliferating cells were found to express Runx2²³, a marker for osteoblast commitment (Supplementary FIG. S8), supporting previous notions that lineage-restricted progenitor cells contribute to the digit bone regeneration^{5,6}. In cKO mice, however Runx2⁺ progenitors and Sp7+ osteoblasts were not induced to proliferate, and the expression of Bmp4, functionally critical for digit bone regeneration⁸, was missing in cKO digits (Supplementary FIG. S8). Furthermore, nerves that are vital for regeneration of rodent digits²⁴ and amphibian limbs²⁵ are located in the proliferative Runx2⁺ mesenchyme close to the Wnt-active nail epithelial cells in control mice, whereas nerves did not extend to the regeneration area close to the epithelium in cKO mice (FIG. 1 h, Supplementary FIG. S9). Moreover, Semaphorin 5a (Sema5a), an axon guidance molecule²⁶, is upregulated in control nail epithelium at 3 weeks after amputation, but not in that of cKO (Supplementary FIG. S10). This may suggest that nerves are attracted to paracrine factor(s) secreted from the Wnt-active nail epithelium, reminiscent of the ability of Wnt-active epithelium to attract nerves, as in the embryonic epidermis²⁷.

To investigate how Wnt-dependent innervations can promote digit regeneration, the present inventors surgically removed nerves before amputation and observed that such intervention led to a suppression of blastema growth similar to that in cKO mice (Supplementary FIG. S11). Subsequent microarray analysis revealed that fibroblast growth factor (FGF) signaling was significantly downregulated in denervated digits at 3 weeks after amputation when blastema grows in control digits. This is particularly interesting, given the vital roles of FGF signaling during amphibian limb regeneration²⁸. Immunostaining confirmed that FGF2 was induced in a distal area of regenerating nail epithelium by 3 weeks after amputation (Supplementary FIG. S12). In contrast, FGF2 was not expressed in the nail epithelium of denervated digits (Supplementary FIG. S12). Notably, cKO mice that showed deficient innervations in the blastema also lacked FGF2 expression in the nail epithelium (Supplementary FIG. S12). Quantitative RT-PCR revealed that fgf receptor 1 (fgfr 1) was expressed in the mesenchymal blastema rather than the regenerating nail epithelium (Supplementary FIG. S12). Consistently, phospho-ERK, a downstream mediator of FGF signaling, is detected in the Runx2⁺ mesenchymal cells of control digits, but not in that of denervated digits and cKO digits (Supplementary FIG. S12). Similar defects in innervation and FGF2/p-ERK induction were observed upon deletion of Wls in K14+ epithelial cells, causing failure in nail and digit regeneration (Supplementary FIG. S13).

To test the function of FGF2 signal within blastema, the present inventors harvested blastema from control mice and allowed their outgrowth in vitro. Addition of FGF2 significantly enhanced the proliferation of blastema cells, while this effect is neutralized by RNA interference against fgfr 1 (Supplementary FIG. S14). Upon exposing blastema cells to bone differentiation media, alizarin red staining became positive, confirming their potential to differentiate into bone (Supplementary FIG. S14). Additionally, implantation of FGF2 soaked beads into denervated digits in vivo induced proliferation of Runx2⁺ mesenchymal blastema, unlike that of control PBS soaked beads (Supplementary FIG. S14).

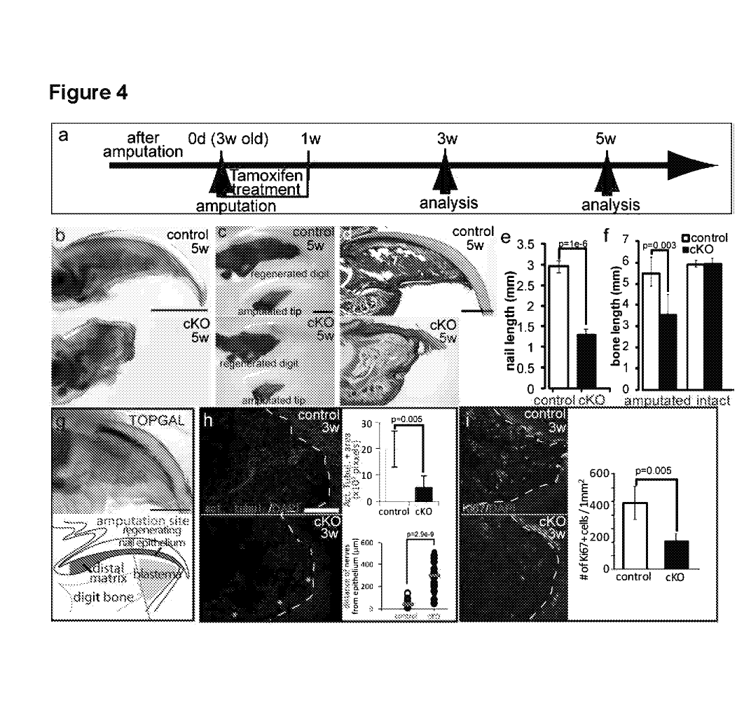

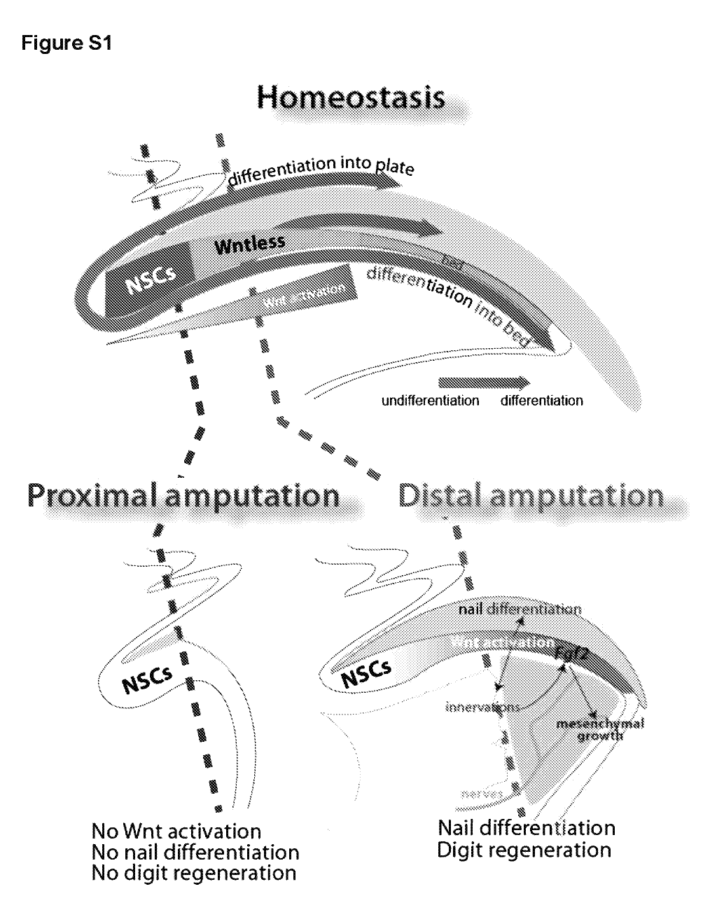

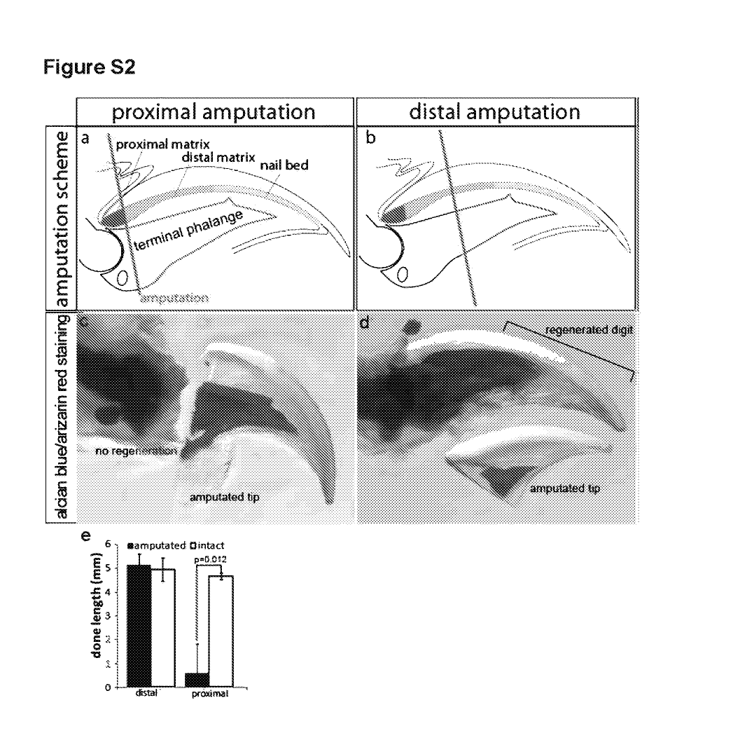

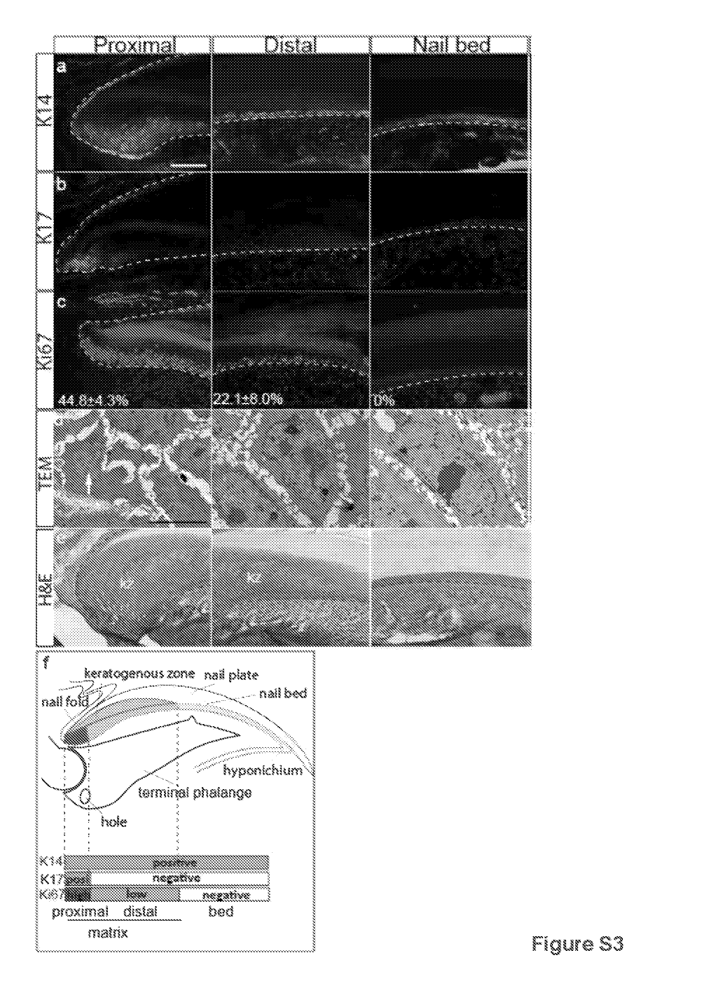

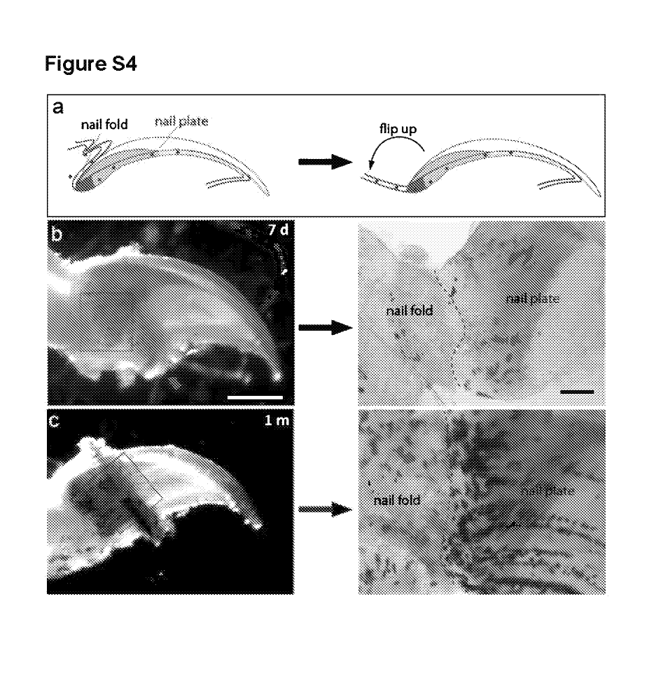

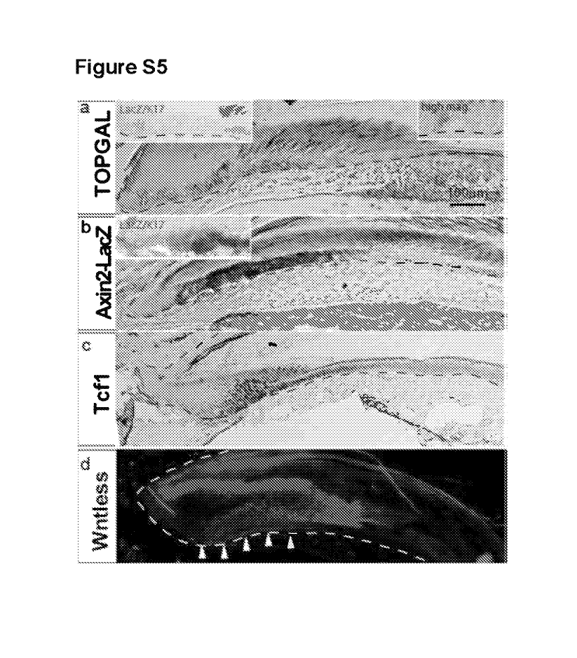

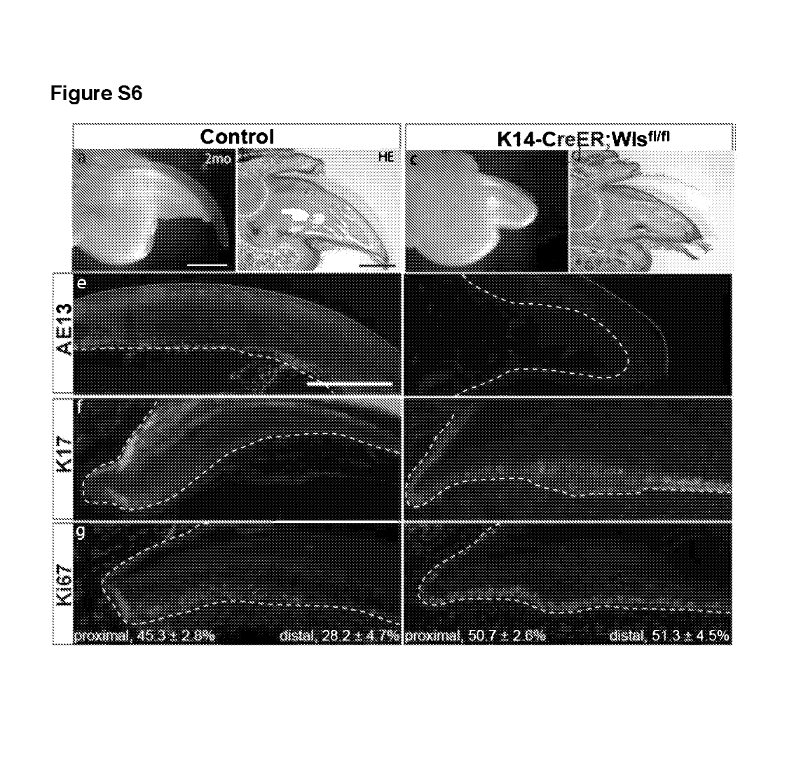

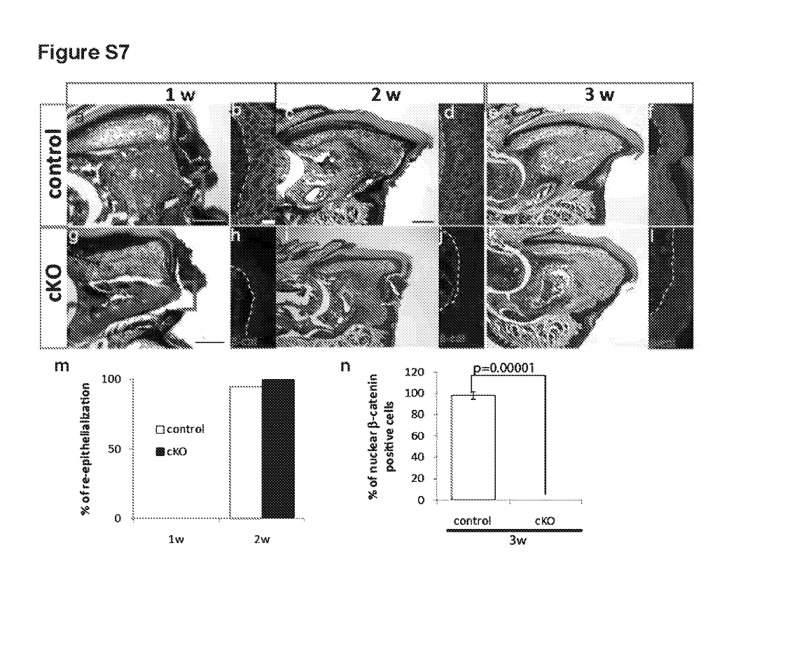

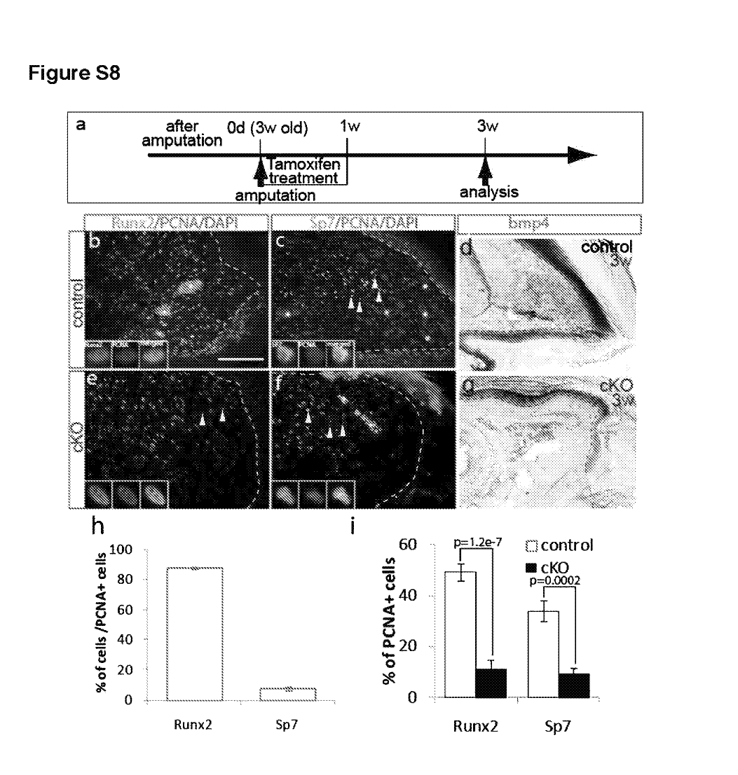

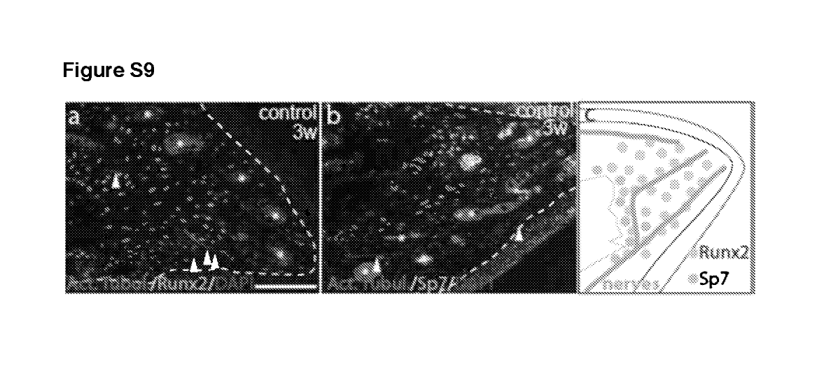

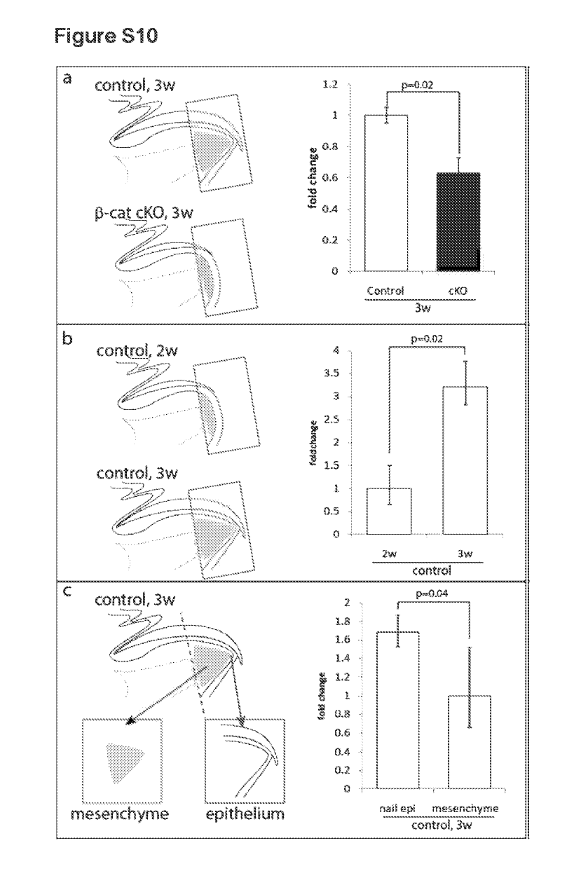

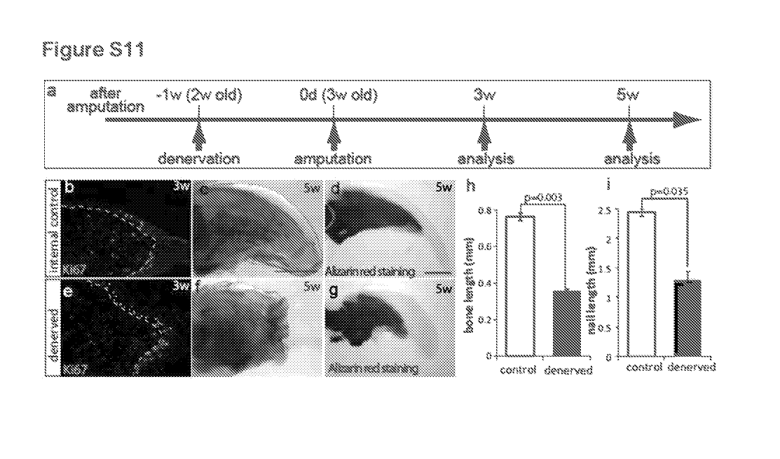

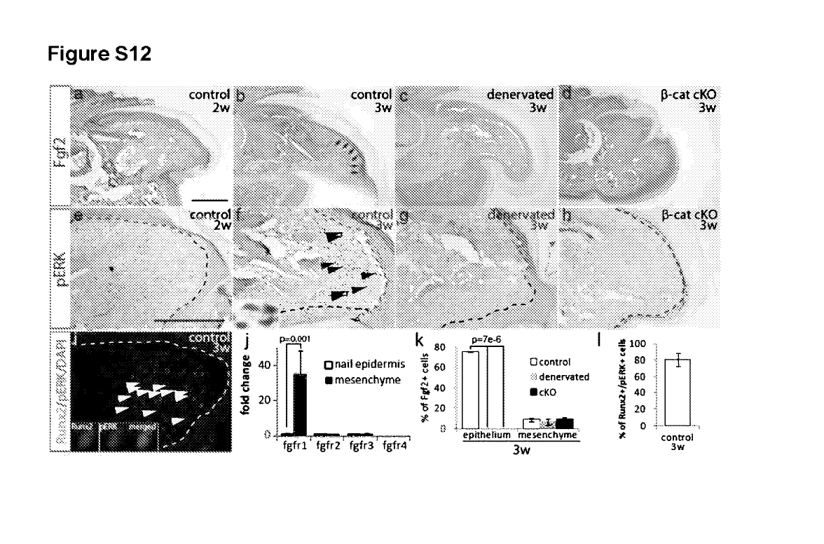

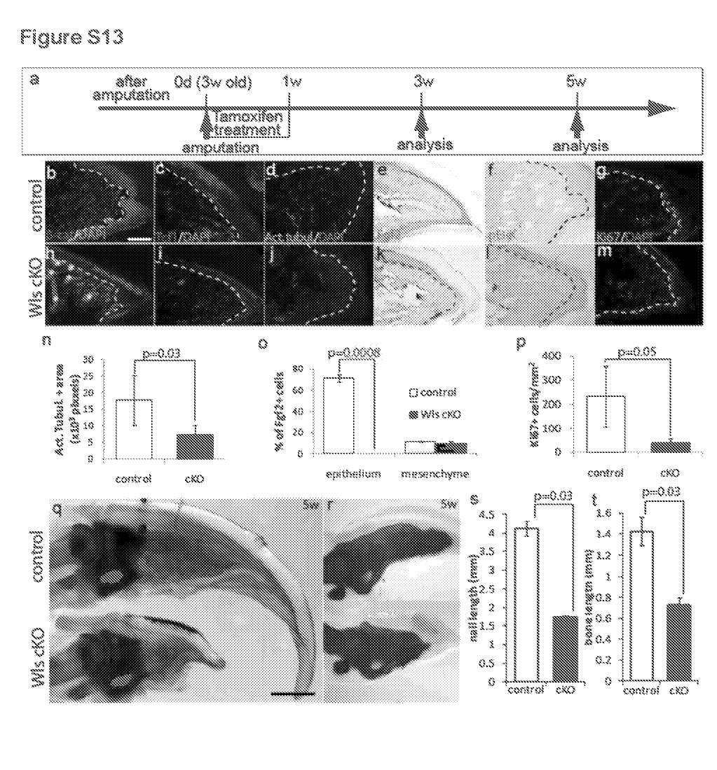

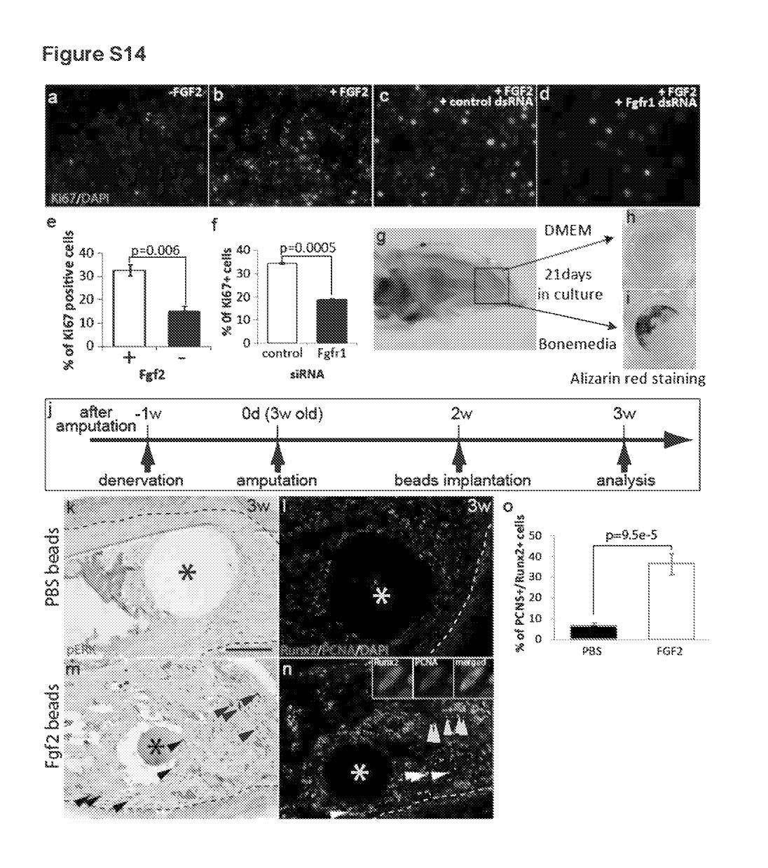

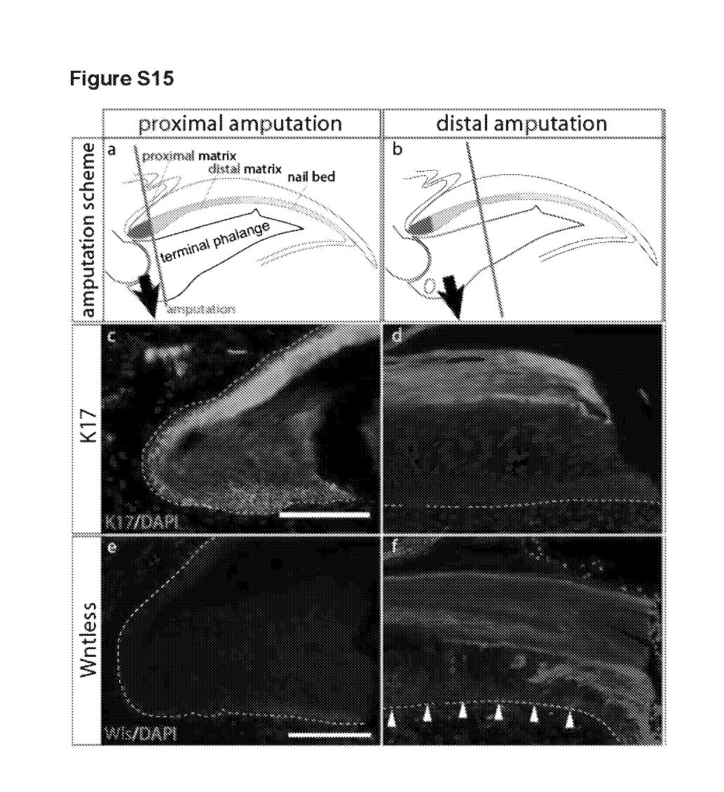

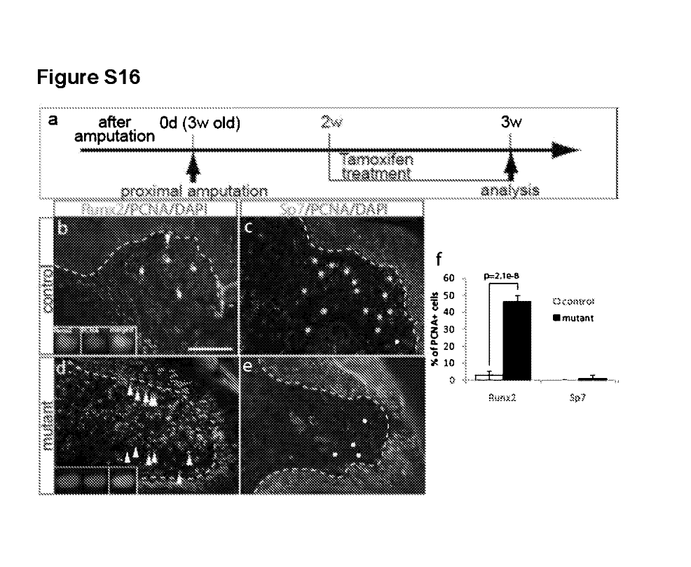

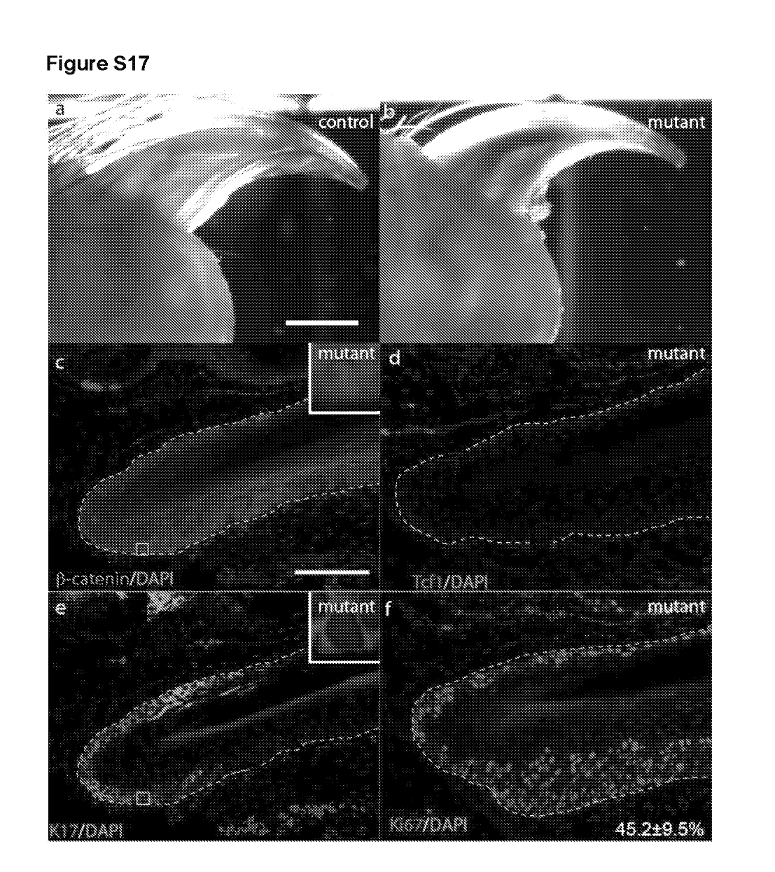

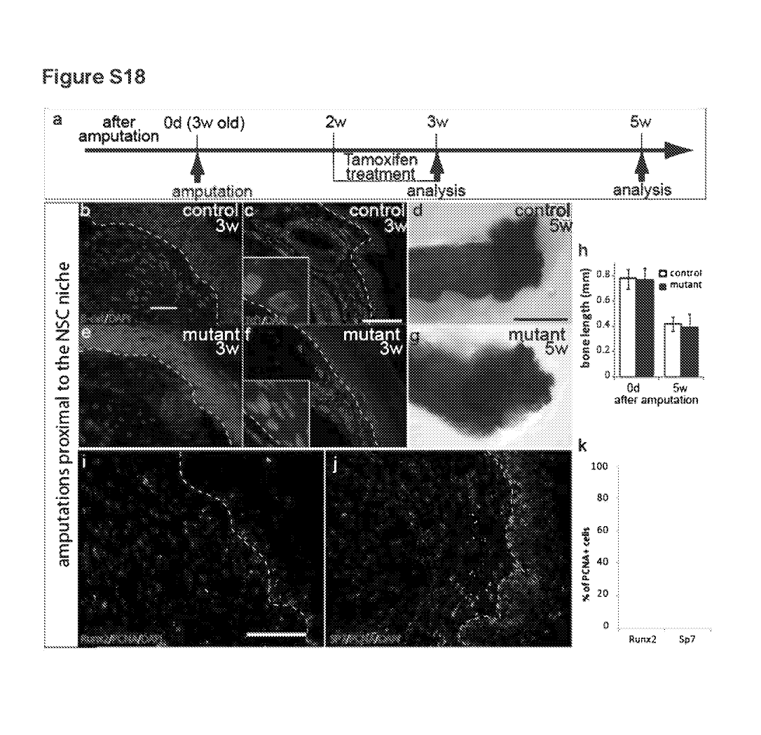

The above results suggest that Wnt activation in the nail epithelium performs dual functions to promote both nail regeneration and Runx2⁺ mesenchymal cell growth through its ability to induce nerve-dependent FGF2 expression. The present inventors then asked why digits do not regenerate after amputations proximal to the nail (Supplementary FIG. S2). Careful examination of the amputated digits showed that amputations of the visible nail plate (i.e., removal of more than 50% of distal phalanx) do not remove the entire NSC region although it is known to fail in regeneration¹⁰ (Supplementary FIG. S15). Unlike distal amputations that induce regeneration, these amputations within the NSC region removed the distal matrix expressing Wntless which is required for initiation of Wnt signaling (Supplementary FIG. S15). Consequently, these amputations failed to activate epithelial Wnt signaling, as revealed by the lack of nuclear β-catenin and TCF1 expression after re-epithelialization (FIG. 4b-c), resulting in the failure to regenerate the nail and digit (FIG. 4g, Supplementary FIG. S2).

To test whether stabilization of β-catenin in K14+ epithelium, including the NSC region, can induce digit regeneration, the present inventors treated K14-CreER;β-catenin^{fl/ex3} mice with TAM following completion of re-epithelialization (FIG. 4a). One week after the initial TAM treatment, basal nail epithelial cells including NSC region exhibited nuclear β-catenin (FIG. 4b). In these tissues, NSC progeny expressed TCF1 as they regenerate distal matrix whereas the proximal end of the NSC region contained TCF1 negative cells (FIG. 4c). Although a transcriptional response to β-catenin stabilization was not directly evaluated, the spatially restricted pattern of TCF1 expression suggest that unidentified mechanisms may be present to cause the disparity in the activation of the pathway which acts downstream of β-catenin stabilization. Nevertheless, it was noteworthy that the regeneration of TCF1⁺ distal nail matrix in these mutant mice accompanied the formation of a well-innervated blastema, which is never observed in control mice upon amputation at this proximal level (FIG. 4d). Accordingly, we observed nail epithelial FGF2 expression and proliferating Runx2+mesenchymal cells, leading to digit bone regeneration (FIG. 4e-f, Supplementary FIG. S16). In these mice, nail regeneration was also grossly apparent and nails without amputations did not show any detectable changes (FIG. 4g, 4i, Supplementary FIG. 17). By contrast, when β-catenin stabilization was induced in K14+ skin epithelial cells after amputation proximal to the NSC region and subsequent re-epithelialization, neither TCF1 expression nor nail formation was observed (Supplementary FIG. S18). This suggests that the skin epidermis and NSCs respond differently upon β-catenin stabilization due to differences within the intrinsic lineage and/or underlying mesenchyme. Notably, Runx2+ cells and Sp7+ cells were found in the mesenchyme but did not show proliferative activity, resulting in the failure to regenerate the digit (Supplementary FIG. S18). These results show that the distally-restricted capacity of digit regeneration is partly due to insufficient Wnt induced signals/mechanisms in the nail epithelium, rather than an inherent absence of cells competent to regenerate the digit bone.

By demonstrating the presence of NSCs that undergo Wnt-dependent differentiation into the nail, the present inventors have uncovered a unique role of the nail epithelium in digit tip regeneration. Past studies in lower vertebrates have documented the vital roles of Wnt and FGF signaling in promoting limb regeneration^{29,30}. These studies were limited by their inability to control gene expression in specific cell populations. The present inventors utilized epithelial-specific gene modification and demonstrated the function of epithelial Wnt signaling in digit tip, thereby opening a new avenue to dissect epithelial-mesenchymal interactions that drive organ regeneration in mammals. The dual function of Wnt signaling in NSC lineage to direct nail formation and digit regeneration appears to be a key mechanism that coordinates regeneration of epithelial and mesenchymal tissues in mammalian digit tip regeneration (Supplementary FIG. S1). The present studies, which reveal aspects of various mechanisms that regulate NSCs and their interactions with mesenchymal cells, have directed the present inventors to propose novel methods for treating amputees.

In order to more clearly set forth the parameters of the present invention, the following definitions are used:

As used in this specification and the appended claims, the singular forms “a”, “an”, and “the” include plural references unless the context clearly dictates otherwise. Thus for example, reference to “the method” includes one or more methods, and/or steps of the type described herein and/or which will become apparent to those persons skilled in the art upon reading this disclosure.

The term “complementary” refers to two DNA strands that exhibit substantial normal base pairing characteristics. Complementary DNA may, however, contain one or more mismatches.

The term “hybridization” refers to the hydrogen bonding that occurs between two complementary DNA strands.

“Nucleic acid” or a “nucleic acid molecule” as used herein refers to any DNA or RNA molecule, either single or double stranded and, if single stranded, the molecule of its complementary sequence in either linear or circular form. In a particular embodiment, the DNA is a cDNA molecule. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it is generally associated in its natural state (i.e., in cells or tissues). An isolated nucleic acid (either DNA or RNA) may further represent a molecule produced directly by biological or synthetic means and separated from other components present during its production.

The term “functional” as used herein implies that the nucleic or amino acid sequence is functional for the recited assay or purpose.

The phrase “consisting essentially of” when referring to a particular nucleotide or amino acid means a sequence having the properties of a given SEQ ID No.: For example, when used in reference to an amino acid sequence, the phrase includes the sequence per se and molecular modifications that would not affect the basic and novel characteristics of the sequence.

A “replicon” is any genetic element, for example, a plasmid, cosmid, bacmid, phage or virus that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression vector” or “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism. Exemplary expression vectors embodied herein comprise nucleic acid sequences (e.g., SEQ ID NOs: 1, 3, 5, 7, 9, 11, 13, 15, and 17) encoding activators of Wnt signaling pathways such as SEQ ID NOs: 2, 4, 6, 8, 10, 12, 14, 16, and 18, respectively.

As used herein, the term “operably linked” refers to a regulatory sequence capable of mediating the expression of a coding sequence and which is placed in a DNA molecule (e.g., an expression vector) in an appropriate position relative to the coding sequence so as to effect expression of the coding sequence. Exemplary coding sequences embodied herein which encode activators of Wnt signaling pathways comprise SEQ ID NOs: 1, 3, 5, 7, 9, 11, 13, 15, and 17. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “oligonucleotide,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to act functionally as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Primers may be labeled fluorescently with 6-carboxyfluorescein (6-FAM). Alternatively primers may be labeled with 4,7,2′,7′-Tetrachloro-6-carboxyfluorescein (TET). Other alternative DNA labeling methods are known in the art and are contemplated to be within the scope of the invention.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity and that may be present, for example, due to incomplete purification, addition of stabilizers, or compounding into, for example, immunogenic preparations or pharmaceutically acceptable preparations. Exemplary isolated proteins embodied herein comprise activators of a Wnt signaling pathway such as, for example, SEQ ID NOs: 2, 4, 6, 8, 10, 12, 14, 16, and 18.

The term “substantially pure” refers to a preparation comprising at least 50-60% by weight of a given material (e.g., nucleic acid, oligonucleotide, protein, etc.). More particularly, the preparation comprises at least 75% by weight, and most particularly 90-95% by weight of the given compound. Purity is measured by methods appropriate for the given compound (e.g. chromatographic methods, agarose or polyacrylamide gel electrophoresis, HPLC analysis, and the like). “Mature protein” or “mature polypeptide” shall mean a polypeptide possessing the sequence of the polypeptide after any processing events that normally occur to the polypeptide during the course of its genesis, such as proteolytic processing from a polypeptide precursor. In designating the sequence or boundaries of a mature protein, the first amino acid of the mature protein sequence is designated as amino acid residue 1.

The term “tag”, “tag sequence” or “protein tag” refers to a chemical moiety, either a nucleotide, oligonucleotide, polynucleotide or an amino acid, peptide or protein or other chemical, that when added to another sequence, provides additional utility or confers useful properties to the sequence, particularly with regard to methods relating to the detection or isolation of the sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by, the trained artisan, and are contemplated to be within the scope of this definition.

The terms “transform”, “transfect”, “transduce”, shall refer to any method or means by which a nucleic acid is introduced into a cell or host organism and may be used interchangeably to convey the same meaning. Such methods include, but are not limited to, viral transduction, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. In other applications, the introduced nucleic acid may exist in the recipient cell or host organism only transiently.

A “clone” or “clonal cell population” is a population of cells derived from a single cell or common ancestor by mitosis.

A “cell line” is a clone of a primary cell or cell population that is capable of stable growth in vitro for many generations.

An “immune response” signifies any reaction produced by an antigen, such as a protein antigen, in a host having a functioning immune system. Immune responses may be either humoral, involving production of immunoglobulins or antibodies, or cellular, involving various types of B and T lymphocytes, dendritic cells, macrophages, antigen presenting cells and the like, or both. Immune responses may also involve the production or elaboration of various effector molecules such as cytokines, lymphokines and the like. Immune responses may be measured both in in vitro and in various cellular or animal systems.

An “antibody” or “antibody molecule” is any immunoglobulin, including antibodies and fragments thereof, that binds to a specific antigen. The term includes polyclonal, monoclonal, chimeric, and bispecific antibodies. As used herein, antibody or antibody molecule contemplates both an intact immunoglobulin molecule and an immunologically active portion of an immunloglobulin molecule such as those portions known in the art as Fab, Fab′, F(ab′)2 and F(v).

The term “about” as used herein refers to a variation in a stated value or indicated amount of up to 10%, 9%, 8%, 7%, 6%, 5%, 4%, 3%, 2%, 1%, 0.75%, 0.5%, 0.25% or 0.1%, wherein the variation can be either an increase or a decrease in the stated value or indicated amount. Use of the term may, therefore, be used to establish a range of values or amounts.

The term exogenous protein may be used to refer to a protein that is not normally expressed in a particular cell or cell type. In an embodiment, a cell or population of cells may be engineered to express a protein via transfection or transduction; under such circumstances, the protein expressed as a result of such engineering is an exogenous protein.

### In Vitro Methods

Methods are described herein for expanding a population of NSCs in vitro and/or generating a population of cells having multipotent regenerative capacity from isolated NSCs in vitro, the method comprising isolating tissue comprising NSCs from a subject, culturing the NSCs in vitro, and activating Wnt signaling in the NSCs, wherein the culturing induces the NSCs to proliferate, thereby expanding the population of NSCs and/or generating the population of cells having multipotent regenerative capacity, and the activating enhances differentiative capacity in the expanded population of NSCs.

The in vitro methods are based on the novel and surprising discoveries of the present inventors. In short, the present inventors have determined that NSCs reside in the proximal nail matrix and can induce regeneration of amputated body parts. The present inventors have, moreover, characterized this population of cells by its high proliferative index (Ki67^{high}) and for expressing the keratins, K14 and K17. Results presented herein demonstrate that these structural and functional features (K14+, K17+, Ki67^{high}) are characteristic of NSCs. The present inventors have, furthermore, found that Wnt signaling contributes to the functional capabilities of NSCs to regenerate amputated body parts, including digits. Indeed, results presented herein demonstrate that NSCs, wherein Wnt signaling pathways are activated, can support and contribute to regeneration of amputated limbs and portions thereof, including components thereof, such as bone, muscle, nerve, skin, and specialized terminal digit structures such as nails. It is noteworthy that the ability to promote innervation is specific to cells of Wnt-activated NSC lineage, as evidenced by the fact that Wnt-activated non-NSC epithelial cells do not induce innervation. The present inventors propose that this special functional activity of cells of Wnt-activated NSC lineage is likely due to their ability to secrete molecules that attract nerve fibers.

Based on these findings, the present inventors devised an in vitro method for expanding a population of NSCs in vitro and/or generating a population of cells having multipotent regenerative capacity from isolated NSCs in vitro, which also provides a method for generating in vitro an expanded population of NSCs that may further comprise administering the expanded population to subjects/patients in need thereof as described herein. The method calls for isolation of tissue comprising NSCs from a subject. Subjects from whence the tissue comprising the nail stem cells is isolated may be any mammal, including without limitation, mice, rats, pigs, dogs, cats, and primates and cadavers of same. More particularly, the tissue may be isolated from a human, including human fetuses and cadavers.

A particular source of tissue comprising NSCs is the nail matrix and, more particularly, the proximal nail matrix. Dissection protocols for the isolation of nail matrix, proximal nail matrix, and distal nail matrix are described herein, as are methods for treating dissected tissue to dissociate cells from the extracellular matrix (ECM) in which they are embedded in the tissue. Such dissociation methods include incubation in 0.25% Trypsin, followed by incubation in 0.35% Collagenase I and DNase I. It is to be understood that other protocols for dissociated of cells are known in the art and can be used in conjunction with the instant methods. It will, moreover, be apparent that isolated NSCs generated in such a manner from nail matrix, irrespective of the matrix type, including proximal nail matrix and distal nail matrix, do not exist in nature since they are normally embedded in ECM of the tissue.

Dissociated cells may be resuspended in DMEM/10% FBS or a media formulation that is approximately equal with regard to cellular nutrients. The percentage of K14+ cells among the dissociated may be determined by cytospin analysis as described below. Cell suspensions containing 1×10⁴ K14⁺ cells may, for example, be cultured with NIH/3T3 feeder layers or other suitable feeder layer (which depends in part on the species from when the K14+ cells are isolated) in F10: DMEM (1:3) media with 10% new born calf serum or other suitable nutritionally equivalent and species appropriate media. NSC colony number and NSC number can be determined using standard means known in the field.

More particularly, after suitable expansion of NSCs in co-culturing conditions with a feeder layer, Wnt signaling pathways can be activated therein and Wnt-activated NSCs can be separated from the epithelial cells using well established methods. See, for example, Rheinwald and Green (1975, Cell 6:331-344, the entire content of which is incorporated herein by reference). Briefly, co-cultures can be exposed to 0.02% EDTA for 15 seconds and pipetted vigorously to remove nearly all of the feeder layer cells. NSC and keratinocyte colonies remain adherent under these conditions and can be disaggregated to single cells by incubating in a solution containing equal parts EDTA and 0.05% trypsin. If required, single cells can be replated together with fresh irradiated feeder cell layers. As disclosed by Rheinwald and Green (supra), adherent colonies may be subcultured when the average colony size reaches about 1000 cells.

In a particular embodiment, the activating is achieved by contacting the nail stem cells with at least one activator of a Wnt signaling pathway. Such activators include, without limitation, canonical Wnt ligands including wnt3, wnt7a, wnt7b, and Wnt10b, which stimulate and initiate Wnt signaling pathways. In another embodiment, the activating is achieved by transducing the nail stem cells with a vector comprising a nucleic acid sequence encoding a Wnt ligand (such wnt3, wnt7a, wnt7b, or Wnt10b), that activates a Wnt signaling pathway in transduced nail stem cells. Exemplary nucleic acid sequences encoding Wnt ligands are known to persons of skill in the art and are, moreover, publicly available via a number of repositories and electronic databases, including GenBank. Exemplary nucleic acid sequences encoding human wnt3a and amino acids therefor can be accessed via, for example, GenBank Accession Nos. BC103921 (SEQ ID NOs: 1 and 2, respectively), BC103922 (SEQ ID NOs: 3 and 4, respectively), and BC103923 (SEQ ID NOs: 5 and 6, respectively). Exemplary nucleic acid sequences encoding human wnt7a and amino acids therefor can be accessed via, for example, GenBank Accession No. BC008811 (SEQ ID NOs: 7 and 8, respectively). Exemplary nucleic acid sequences encoding human wnt7b and amino acids therefor can be accessed via, for example, GenBank Accession Nos. NM_{—}058238 XM_{—}001718758 (SEQ ID NOs: 9 and 10, respectively), and BC034923 (SEQ ID NOs: 11 and 12, respectively). Exemplary nucleic acid sequences encoding human wnt10a and amino acids therefor can be accessed via, for example, GenBank Accession Nos. BC034352 (SEQ ID NOs: 13 and 14, respectively), NM_{—}025216 (SEQ ID NOs: 15 and 16, respectively), and BC052234 (SEQ ID NOs: 17 and 18, respectively).

For Wnt activation using recombinant wnt ligand protein, concentrations of about 0.1 μM-100 μM may be used. For transfection, NSCs will be transfected with 0 or 2 mg pCAGGS-Wnt10b (or other canonical wnt ligand) vector using Lipofectamine.

It is to be understood that supplementation with standard media additives for prevention of bacterial or fungal infection (such as, e.g., penicillin-streptomycin) is not precluded from the method of the present invention. It is to be further understood that methods for expanding NSCs may also include additional growth factor and/or cytokine supplementation.

The method for expanding a population of nail stem cells in vitro and/or generating a population of cells having multipotent regenerative capacity from isolated nail stem cells in vitro may further comprise administering the expanded population of nail stem cells or the population of cells having multipotent regenerative capacity to a recipient in need thereof. For such applications, Wnt activation may particularly be effected after expansion of the NSC population and immediately before administering the Wnt-activated NSCs to the recipient. Recipients in need thereof may be amputees, wherein an entire limb or a portion thereof, including specialized distal ends, have been removed, or subjects afflicted with, for example, nail dystrophy or nail psoriasis in which nail growth and formation are damaged. In circumstances in which the donor (subject from whom the tissue comprising the nail stem cells is isolated) and the recipient are the same individual, the nail stem cell transplant may be referred to as an autologous transplant. In circumstances wherein the donor and recipient are different individuals of the same species, the nail stem cell transplant may be referred to as an allogeneic transplant.

### Agents

As used herein, an “agent”, “candidate compound”, or “test compound” may be used to refer to, for example, nucleic acids (e.g., DNA and RNA), carbohydrates, lipids, proteins, peptides, peptidomimetics, small molecules and other drugs. More particularly an agent may refer to short hairpin RNA (shRNA), small interfering RNA (siRNA), neutralizing and/or blocking antibodies, or molecules known to promote muscle and bone cell proliferation and/or differentiation and neuronal plasticity and/or growth cone guidance.

A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound.

Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene.

As described herein, an agent identified using the method of the present invention that is a “modulator of NSC proliferation” is defined as an agent that is capable of modulating (e.g., increasing or decreasing) in vitro proliferation of nail stem cells. Such an agent may be identified by its ability to effect a change in the number NSCs. Promoters of NSC proliferation identified in screening methods described herein are identified by their ability to increase the number of NSCs in a culture relative to NSCs cultured in the presence of a control agent or no agent. Agents that act as modulators of NSC differentiation may also be identified using screening methods described herein. Such agents may modulate the expression of a NSC marker, such as K14 or K17, in a population of K14+ cells incubated in accordance with methods described herein.

A change effected by an agent that is a modulator of NSC proliferation or differentiation is determined relative to that of a population of K14+ cells incubated in parallel in the absence of the agent or in the presence of a control agent (as described below), either of which is analogous to a negative control condition.

The term “control substance”, “control agent”, or “control compound” as used herein refers a molecule that is inert or has no activity relating to an ability to modulate a biological activity. With respect to the present invention, such control substances are inert with respect to an ability to modulate NSC proliferation or differentiation in vitro. Exemplary controls include, but are not limited to, solutions comprising physiological salt concentrations.

It is to be understood that agents capable of modulating NSC proliferation or differentiation, as determined using in vitro methods described herein, are likely to exhibit similar modulatory capacity in applications in vivo.

Modulatory agents identified using the screening methods of the present invention and compositions thereof can thus be administered for therapeutic treatments. In therapeutic applications, modulatory agents that promote NSC proliferation or differentiation (i.e., promoters of NSC proliferation or differentiation) and compositions thereof are administered to an amputee, a patient from which a body part has been amputated (e.g., limb or portion of a limb, including a digit tip), or to a patient suffering from nail dystrophy or nail psoriasis in an amount sufficient to at least partially regenerate the amputated body part or arrest a symptom or symptoms of the disease and its complications, respectively. An amount adequate to accomplish this is defined as a “therapeutically effective amount or dose.” It is also envisioned that modulatory agents may also be administered in combination with NSCs to treat amputees or patients suffering from nail dystrophy or nail psoriasis. Amounts effective for this use will depend on the severity of the disease and the weight and general state of the patient.

### Methods for Determining Expression Levels of NSC Cell Markers

Based on the guidance presented herein and knowledge in the relevant scientific fields, the expression level of a cellular marker of NSCs can be determined using a variety of techniques. Exemplary markers of NSCs include, but are not limited to, K14, K17, and high levels of Ki67^{high}. As described herein, NSCs are characterized as K14+K17+ Gli2−. Expression levels of such markers (either a positive or a negative marker) may be assessed with respect to expressed nucleic acid corresponding to a cell marker (e.g., mRNA, total RNA) or with respect to polypeptides encoded by same. A variety of standard protocols may be used to determine, for example, RNA level, including, but not limited to: polymerase chain amplification and detection of amplified products therefrom, ribonuclease protection (RNase protection) assay, and Northern blot analysis. The principles and general procedures of each of these methods are, moreover, known in the art. In a particular embodiment of the invention, real-time PCR is used to detect gene expression of NSC markers.

A variety of protocols are available for measuring and/or detecting expression levels of polypeptides. Protocols for detecting polypeptide expression, such as, for example, immunohistochemistry and immunoblotting, are known in the art. These protocols are generally applicable to detecting K14, K17, Ki67, and Gli2 polypeptides. Particular methods for detecting K14, K17, Ki67, and Gli2 polypeptides are described in the Examples presented herein, as are reagents for performing such methods.

In general, immunoassays for polypeptides typically comprise contacting a sample, such as a population of cells (e.g., incubated in NSC population proliferative promoting conditions or lysates thereof) in the presence of an antibody that specifically or selectively binds to a polypeptide in question, e.g., a detectably labeled antibody capable of identifying the particular polypeptide and detecting the bound antibody by any of a number of techniques well-known in the art (e.g., Western blot, ELISA, FACS).

The biological sample may be brought in contact with and immobilized onto a solid phase support or carrier such as nitrocellulose, or other solid support that is capable of immobilizing cells, cell particles or soluble proteins. The support may then be washed with suitable buffers followed by treatment with the detectably labeled antibody that selectively or specifically binds to the particular polypeptide (e.g., a NSC marker). The solid phase support may then be washed with the buffer a second time to remove unbound antibody. The amount of bound label on a solid support may then be detected by conventional means.

More particularly, NSC marker protein levels can be assessed by cell surface staining; ELISA; intracellular staining (e.g., K14 and K17); and Western Blot.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Particular supports include polystyrene beads. Those skilled in the art will know many other suitable carriers for binding antibody or antigen, or will be able to ascertain the same by use of routine experimentation.

An antibody can be detectably labeled by linking same to an enzyme and using the labeled antibody in an enzyme immunoassay (EIA) (Voller, A., “The Enzyme Linked Immunosorbent Assay (ELISA)”, 1978, Diagnostic Horizons 2:1, Microbiological Associates Quarterly Publication, Walkersville, Md.); Voller, A. et al., 1978, J. Clin. Pathol. 31: 507-520; Butler, J. E., 1981, Meth. Enzymol. 73:482; Maggio, E. (ed.), 1980, Enzyme Immunoassay, CRC Press, Boca Raton, Fla.; Ishikawa, E. et al., (eds.), 1981, Enzyme Immunoassay, Kgaku Shoin, Tokyo). The enzyme that is bound to the antibody reacts with an appropriate substrate, particularly a chromogenic substrate, in such a manner as to produce a chemical moiety detectable, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect a polypeptide through the use of a radioimmunoassay (MA). The radioactive isotope can be detected by such means as the use of a gamma counter or a scintillation counter or by autoradiography.

An antibody may also be labeled with a fluorescent compound. When the fluorescently labeled antibody is exposed to light of the proper wavelength, its presence can be detected due to fluorescence emission. Among the most commonly used fluorescent labeling compounds are fluorescein isothiocyanate, rhodamine, phycoerythrin, phycocyanin, allophycocyanin, o-phthaldehyde and fluorescamine.

An antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

An antibody can also be detectably labeled by coupling it to a chemiluminescent compound. The presence of the chemiluminescent-tagged antibody is determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label an antibody. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

The basic molecular biology techniques used to practice the methods of the invention are well known in the art, and are described for example in Sambrook et al., 1989, Molecular Cloning: A Laboratory Manual, Cold Spring Harbor Laboratory, New York; Ausubel et al., 1988, Current Protocols in Molecular Biology, John Wiley & Sons, New York; and Ausubel et al., 2002, Short Protocols in Molecular Biology John Wiley & Sons, New York).

### Agents Identified by the Screening Methods of the Invention

The invention provides methods for identifying agents (e.g., candidate compounds or test compounds) that modulate (promote or inhibit) NSC proliferation, survival, and/or differentiation. Agents that are capable of promoting NSC proliferation and/or survival, for example, as identified by a screening method described herein, are useful as candidate pro-regenerative therapeutics.

A list of conditions and disorders that may be treated using an agent identified using a method of the invention includes, without limitation: amputations of a limb (e.g., an arm or leg) or unit or portion thereof, including digits (e.g., fingers and toes) and portions thereof, particularly digit tips; nail dystrophy; and nail psoriasis.

Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233, each of which is incorporated herein in its entirety by reference.

Libraries of compounds may be presented, e.g., presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (19900 Science 249:386-390; Devlin (1990) Science 249:404-406; Cwirla et al. (1990) Proc. Natl. Acad. Sci. USA 87:6378-6382; and Felici (1991) J. Mol. Biol. 222:301-310), each of which is incorporated herein in its entirety by reference.

### Therapeutic Uses of Agents Identified

The invention provides for treatment of patients with amputations (amputees), nail dystrophy, or nail psoriasis by administration of a therapeutic agent identified using the above-described methods. Such agents include, but are not limited to proteins, peptides, protein or peptide derivatives or analogs, antibodies, nucleic acids, and small molecules. Wnt-activated NSCs and compositions thereof are also envisioned as therapeutic agents for administering to patients in need thereof.

The invention provides methods for treating patients afflicted with amputations, nail dystrophy, or nail psoriasis comprising administering to a subject an effective amount of an agent or compound identified by the method of the invention. In a particular aspect, the compound is substantially purified (e.g., substantially free from substances that limit its effect or produce undesired side-effects). The subject is particularly an animal, including but not limited to animals such as cows, pigs, horses, chickens, cats, dogs, etc., and is more particularly a mammal, and most particularly a human. In a specific embodiment, a non-human mammal is the subject.

Formulations and methods of administration that can be employed when the compound comprises a nucleic acid are described above; additional appropriate formulations and routes of administration are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu (1987) J. Biol. Chem. 262:4429-4432), and construction of a nucleic acid as part of a retroviral or other vector. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally, e.g., by local infusion during surgery, topical application, e.g., by injection, by means of a catheter, or by means of an implant, said implant being of a porous, non-porous, or gelatinous material, including membranes, such as sialastic membranes, or fibers.

In another embodiment, the compound can be delivered in a vesicle, in particular a liposome (see Langer (1990) Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton (1987) CRC Crit. Ref. Biomed. Eng. 14:201; Buchwald et al. (1980) Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target, e.g., a transplant site, thus requiring only a fraction of the systemic dose (see, e.g., Goodson, in Medical Applications of Controlled Release, supra, vol. 2, pp. 115-138 (1984)). Other controlled release systems are discussed in the review by Langer (1990, Science 249:1527-1533).

### Pharmaceutical Compositions

The present invention also provides pharmaceutical compositions. Such compositions comprise a therapeutically effective amount of an agent and a pharmaceutically acceptable carrier. In a particular embodiment, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin, incorporated in its entirety by reference herein. Such compositions will contain a therapeutically effective amount of the compound, preferably in purified form, together with a suitable amount of carrier so as to provide the form for proper administration to the subject. The formulation should suit the mode of administration.

In a particular embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment of patients with amputations (amputees), nail dystrophy, or nail psoriasis can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Suppositories generally contain active ingredient in the range of 0.5% to 10% by weight; oral formulations preferably contain 10% to 95% active ingredient. Effective doses may be extrapolated from dose-response curves derived from in vitro or animal model test systems.

It is to be understood that pharmaceutical compositions may comprise NSCs in combination with agents identified in the screening assays described herein. Such pharmaceutical compositions may further comprise activators of Wnt signaling pathways.

### Nucleic Acids

The invention provides methods of identifying agents capable of modulating NSC proliferation, survival, and/or differentiation. Accordingly, the invention encompasses administration of a nucleic acid encoding a peptide or protein capable of modulating NSC proliferation, survival, and/or differentiation, as well as antisense sequences or catalytic RNAs capable of promoting NSC proliferation, survival, and/or differentiation.

Any suitable methods for administering a nucleic acid sequence available in the art can be used according to the present invention.

Methods for administering and expressing a nucleic acid sequence are generally known in the area of gene therapy. For general reviews of the methods of gene therapy, see Goldspiel et al. (1993) Clinical Pharmacy 12:488-505; Wu and Wu (1991) Biotherapy 3:87-95; Tolstoshev (1993) Ann. Rev. Pharmacol. Toxicol. 32:573-596; Mulligan (1993) Science 260:926-932; and Morgan and Anderson (1993) Ann. Rev. Biochem. 62:191-217; May (1993) TIBTECH 11(5): 155-215. Methods commonly known in the art of recombinant DNA technology which can be used in the present invention are described in Ausubel et al. (eds.), 1993, Current Protocols in Molecular Biology, John Wiley & Sons, NY; and Kriegler (1990) Gene Transfer and Expression, A Laboratory Manual, Stockton Press, NY.

In a particular aspect, the compound comprises a nucleic acid encoding a peptide or protein capable of modulating NSC proliferation, survival, and/or differentiation, such nucleic acid being part of an expression vector that expresses the peptide or protein in a suitable host. In particular, such a nucleic acid has a promoter operably linked to the coding region, said promoter being inducible or constitutive (and, optionally, tissue-specific). In another particular embodiment, a nucleic acid molecule is used in which the coding sequences and any other desired sequences are flanked by regions that promote homologous recombination at a desired site in the genome, thus providing for intrachromosomal expression of the nucleic acid (Koller and Smithies (1989) Proc. Natl. Acad. Sci. USA 86:8932-8935; Zijlstra et al. (1989) Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject, known as “ex vivo gene therapy”. NSCs could, for example, be transformed in vitro with a nucleic acid sequence that encodes a polypeptide that confers constitutive Wnt activation, such as, for example, stabilized β-catenin and then transplanted into a subject in need thereof.

In another embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors.

In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al. (1989) Nature 342:435-438).

In a further embodiment, a retroviral vector can be used (see Miller et al. (1993) Meth. Enzymol. 217:581-599). These retroviral vectors have been modified to delete retroviral sequences that are not necessary for packaging of the viral genome and integration into host cell DNA. The nucleic acid encoding a desired polypeptide to be used in gene therapy is cloned into the vector, which facilitates delivery of the gene into a subject. More detail about retroviral vectors can be found in Boesen et al. (1994) Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al. (1994) J. Clin. Invest. 93:644-651; Kiem et al. (1994) Blood 83:1467-1473; Salmons and Gunzberg (1993) Human Gene Therapy 4:129-141; and Grossman and Wilson (1993) Curr. Opin. in Genetics and Devel. 3:110-114.

Adenoviruses may also be used effectively in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson (1993) Current Opinion in Genetics and Development 3:499-503 present a review of adenovirus-based gene therapy. Bout et al. (1994) Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al. (1991) Science 252:431-434; Rosenfeld et al. (1992) Cell 68:143-155; Mastrangeli et al. (1993) J. Clin. Invest. 91:225-234; PCT Publication WO94/12649; and Wang, et al. (1995) Gene Therapy 2:775-783. Adeno-associated virus (AAV) has also been proposed for use in gene therapy (Walsh et al. (1993) Proc. Soc. Exp. Biol. Med. 204:289-300; U.S. Pat. No. 5,436,146).

Another suitable approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr (1993) Meth. Enzymol. 217:599-618; Cohen et al. (1993) Meth. Enzymol. 217:618-644; Cline (1985) Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. In a particular embodiment, epithelial cells are injected, e.g., subcutaneously. In another embodiment, recombinant skin cells may be applied as a skin graft onto the subject; recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to NSCs, neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver. In a particular embodiment, the cell used for gene therapy is autologous to the subject that is treated.

In another embodiment, the nucleic acid to be introduced for purposes of gene therapy may comprise an inducible promoter operably linked to the coding region, such that expression of the nucleic acid is controllable by adjusting the concentration of an appropriate inducer of transcription.

Direct injection of a DNA coding for a peptide or protein capable of modulating NSC proliferation, survival, and/or differentiation may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection.

### Homogeneous Populations of NSCs

The novel methods of the present invention facilitate the generation of a homogeneous population of NSCs comprising about or at least 10³-10⁹ homogeneous K14+, K17+ NSCs, wherein the K14+, K17+ NSCs do not express cellular markers particular to distal nail matrix, such as Gli2. More specifically, the isolated homogeneous population of K14+, K17+ NSCs generated from K14+ proximal nail matrix do not express cellular markers characteristic of and particular to cells isolated from the distal nail matrix or nail bed cells that are negative for K17. Accordingly, a homogeneous population of K14+, K17+ NSCs of the present invention does not include cells that express, for example, Gli2.

The isolated population of about or at least 10³-10⁹ homogeneous K14+, K17+ NSCs generated from K14+ proximal nail matrix as described herein is, therefore, homogeneous with respect to the expression of only those cellular markers characteristic of proximal matrix NSCs. Exemplary markers of proximal matrix NSCs include expression of K14 and K17, which combination of expression is indicative of proximal nail matrix NSCs.

Prior to the present method, an isolated, homogeneous population of about or at least about 10³-10⁹ homogeneous K14+, K17+ NSCs, wherein the NSCs do not express cellular markers of distal nail matrix cells and are purified therefrom, had not been generated.

Unless defined otherwise, all technical and scientific terms used herein have the same meaning as commonly understood by one of ordinary skill in the art to which this invention belongs.

It is to be understood that this invention is not limited to particular assay methods, or test agents and experimental conditions described, as such methods and agents may vary. It is also to be understood that the terminology used herein is for the purpose of describing particular embodiments only, and is not intended to be limiting, since the scope of the present invention will be limited only the appended claims.

### Example I

### Methods and Materials

Mice and Sample Collections

All mice except β-catenin^{fl/ex3} mice³¹ were obtained from Jackson Laboratories, and maintained in the Smilow Central Animal Facility at NYU Langone Medical Center. All animal protocols were approved by the IACUC at NYU School of Medicine. Cre recombination in K14-CreER; Rosa^{stop-LacZ 32}, K14-CreER;β-catenin^{fl/fl 33}, K14-CreER;β-catenin^{fl/ex3} and K14-CreER;Wntless^{fl/fl 34} mice was induced by TAM injection as published¹⁴. For nail sample collections, we sacrificed mice by CO₂ narcosis, and harvested the middle 3 digits of the hind limbs.

X-Gal Staining

Nail samples from K14-CreER; Rosa^{stop-LacZ}, Topgal³⁵ and Axin2-LacZ³⁶ mice were fixed in 4% PFA at 4° C. for 30 min, rinsed with PBS and incubated in X-gal (5-bromo-4-chloro-3-indolyl-β-d-galactopyranoside) solution as previously described¹⁴. After photographing X-gal stained whole mount nail samples under a dissection microscope (Zeiss, Discovery V12), nail samples were incubated in 30% sucrose at 4° C. overnight, embedded into OCT-compound (Sakura), and cut into 10 μm thick frozen sections.

Immunohistochemistry

Nails were fixed in 10% Zinc buffered formalin at 4° C. 2 nights, and washed in PBS twice. After decalcification in 22.5% formic acid/10% sodium citrate buffer at RT for 2 hr, nails were dehydrated through ethanol and xylene, embedded in paraffin, and cut into 6 μm sections. Following rehydration, paraffin-sectioned tissues were processed in hematoxylin and eosin, or Masson's trichrome staining. For immunohistochemistry, antigen retrieval was performed by microwaving sections for 6 min on high-wattage setting in 1× TE buffer (pH. 8.0). Sections were blocked in 10% fetal bovine serum (FBS)/PBS at RT for 1 hr, then incubated with primary antibodies against K14 (1:500, Covance), K17 (1:500, Abcam), AE13 (1:50, a gift of TT Sun, New York University), Ki67 (1:50, Abcam), Ctnnb1 (1; 400, Sigma), Tcf1 (1:50, Cell signaling), Runx2 (1:100, Sigma), Sp7 (1:100, Santa Cruz)acetylated tubulin (1:500, Sigma), Fgf2 (1:100, Santa Cruz), pERK (1:100, Cell signaling; 1:20, Abcam) and Msx1 (1:20, Abcam) at 4° C. overnight, followed by incubation with fluorescein conjugated, or biotinylated secondary antibodies at RT for 2 hr. For biotinylated secondary antibodies, a third amplification step with streptavidin-conjugated TRITC (1:200, Vector) or Horseradish peroxidase (HRP, 1:500, Upstate) was performed. A diaminobenzidine (DAB) substrate solution (Sigma) was used for developing signals for HRP. All antibodies were diluted in 0.1% Triton-X 100/PBS.

Transmission Electron Microscopy

Samples were fixed in 0.1M sodium cacodylate buffer (pH 7.2) containing 2.5% glutaraldehyde, and 2% paraformaldehyde for 2 h at RT and 4° C., O/N. After post-fixation in 1% osmium tetroxide for 1.5 h at RT, samples were processed using standard methods and embedded in EMbed 812 (Electron Microscopy Sciences). Semi-thin sections were cut at 1 um and stained with 1% Toluidine Blue to evaluate the quality of preservation. Ultrathin sections (60 nm) were cut, mounted on copper grids and stained with uranyl acetate and lead citrate. Stained grids were examined under a Philips CM-12 electron microscope (FEI; Eindhoven, Netherlands) and photographed with a Gatan (4 k×2.7 k) digital camera (Gatan, Inc.).

Whole-Mount Visualization of Digit Bone

Nails were fixed in 4% PFA at 4° C., O/N. After washing in 1% KOH in H₂O, digits were incubated serially in 20% glycerol/1% KOH for 3-6 h at RT, 50% glycerol/1% KOH for 4-16 h at RT and 100% glycerol 0/N at RT.

Immunocytochemistry

Dissociated cells were resuspended in 1% FBS/PBS and spun onto glass slides using Cytospin 3 (Shandon, Cheshire, UK). The slide was fixed with acetone at −20° C. for 10 min. Following washes in 1×PBS, slides were blocked in 10% fetal bovine serum (FBS)/PBS at RT for 1 h, then incubated with primary antibodies against K14 (1:500, Covance) at 4° C., O/N, followed by incubation with AlexaFlor 488 conjugated secondary antibody at RT for 2 h. After washing in 1×PBS, slides were incubated with primary antibodies against K17 (1:5000, Abcam) at 4° C., O/N, and biotinylated secondary antibodies at RT for 2 h, and then streptavidin-labeled tetramethyl rhodamine isothiocyanate (SA-TRITC) (1:200, Vector) at RT for 1 h. Primary antibodies were diluted in 10% FBS/PBS, and secondary antibodies were diluted in PBS.

Colony Forming Assay

Thirty nails from at least 5 different mice (8-10 week-old FVB mice) were collected, and the nail fold overhanging the nail plate was removed with surgical blades and forceps under a dissection microscope. Dissected fragments were incubated in 0.25% Trypsin for 1 h 45 min at 37° C., and then in 0.35% Collagenase I and DNase I for 10 min each at 37° C. Dissociated cells were resuspended in DMEM/10% FBS. The percentage of K14⁺ cells was then determined by cytospin analysis as described below. Cell suspensions containing 1×10⁴ K14⁺ cells were cultured with NIH/3T3 feeder layers (a gift from Dr. Alka Mansukhani, New York University) in F10:DMEM (1:3) media with 10% new born calf serum in 6-well plates³⁷. After 14 days in culture, cells were fixed with 10% buffered formalin and stained with 1% Rhodamine B. The number of colonies was manually counted and the size of the colonies was measured using image analysis software (Image J, NIH), and colony forming efficiency (the number of colonies larger than 3 mm²/1×10⁴ cells) was calculated. Studies were performed three independent times.

Gene Expression Profiling of NSCs by Microarray

7-8 week-old K14-rtTA; TetO-H2B-GFP mice (Jackson Laboratory) were treated with Dox for 7 d to label the entire K14⁺ matrix cells with GFP. Thirty digits from at least 5 different mice were collected and single cell suspensions were prepared as described above. The cells were incubated with APC conjugated anti-CD49f antibody in 1% FBS/PBS, for 15 min at RT. Basal nail epithelial cells from each fraction were isolated using FACS based on the GFP label, representing K14 positivity, and expression of CD49f, a general marker of basal cells. To obtain sufficient cells for oligonucleotide gene chip hybridization, we used the Ovation RNA Amplification System V2 (Nugen) for mRNA amplification. The amplified mRNA was labeled and hybridized to the Mouse 430.2 microarrays (Affymetrix). Data was analyzed with GeneSpring X software, and genes that were differentially regulated at least 2 fold were selected for further analysis. GEO accession numbers; GSE45494, GSM1105640, GSM1105641, GSM1105642 and GSM1105643.

Digit Amputation

Digit amputation was performed according to the previously reported method with modification⁸. Briefly, the central three digits (digit 2, 3 and 4) of hind limbs of 21 days old mice were amputated at the level of the middle of nail matrix or in NSCs area. Amputated digits were collected at 1, 2, 3 and 5 weeks after amputation, and processed for Alucian blue/Alizarin red or immunohistochemistry. More than 10 different digits from 5 mice were used for each time point. Studies were repeated three times.

In Situ Hybridization

Digoxigenin-labeled RNA probes complementary to Bmp4 (a gift of Han M and Muneoka K, Tulane University) were synthesized according to manufacturer's instructions (DIG-RNA Labeling Kit, Roche). In situ hybridization was performed using previously described method¹⁴. Studies were repeated three times.

Denervation

The sciatica nerve of 2-week-old mice was approached through a rectilinear longitudinal cutaneous incision on the lateral surface of the right thigh, and a 3-5 mm segment was removed. The wound was closed with surgical staple. Digits were amputated at 1 week after denervation. Amputated digits were collected at 1, 2, 3, 4 and 5 weeks after amputation. More than 10 different digits from 5 mice were used for each time point. Studies were repeated with 3 different litters.

Blastema Cell Culture and Bone Differentiation Assay

Digit tip proximal to the terminal phalanx was collected at 3 w after digit amputation. Mesenchymal blastema cell mass was separated from the nail epidermis by sine forceps and needle under the dissecting microscope. Isolated blastema cell mass was placed in 24-well plate with DMEM (invitrogen)/10% FBS (Cellgro), and incubated at 37° C., 5% CO₂. After 1 w in culture, blastema cells were transfected with 50 nM siRNA against for FGFR1 (Invitrogen, MSS204294 and MSS204295) or control siRNA, using Lipofectamine RNAiMAX (Invitrogen). Transfected cells were incubated in DMEM (invitrogen)/10% FBS (Cellgro) with or without 20 ng/ml FGF2 (Sigm-Aldrich) at 37° C., 5% CO₂ for 2 d, and were stained for Ki67 as described above. For bone differentiation assay, culture media was replaced with HyClone® Advance STEM™ Osteogenesis differentiation medium (Thermo Scientific) at 7 days in culture. After 3 w in culture, mineralization was assessed by alizarin red staining. In brief, the cultures were fixed in 10% Zinc buffered formalin at RT for 10 min, washed in PBS twice, and stained with 2% alizarin red S (Sigma) in distilled water for 5 min at RT. The stained cell layers were washed, rinsed twice with distilled water, and air-dried.

Bead Implantation

The present inventors performed bead implantation experiments using previously described method (Yu et al., 2010) with the following modifications. Briefly, Affi-Gel Blue Gel beads (Bio-Rad) were washed with 0.1% BSA/PBS then soaked with recombinant human Fgf2 (Sigma) at a concentration of 0.3 mg/ml or 0.1% BSA/PBS as a control for 2 hours at room temperature. Bead implantation was performed at 2 weeks after digit amputation after the completion of wound closure was confirmed.

Statistical Analysis

Student's t-test was used to calculate p-values on Microsoft Excel, with two-tailed tests and unequal variance.

### Results

In order to investigate the potential role of the nail epithelium in digit regeneration, the present inventors first sought to locate and identify NSCs. Toward this end, lineage tracing was performed using K14-creER; R26R-lacZ reporter mice. A single injection of tamoxifen (TAM) was used to genetically label a small subset of K14+ nail basal epidermal cells, including nail matrix cells and bed cells, with LacZ (FIG. 1b, c). Over time, descendants of the labeled K14+ nail epithelial cells extended linearly and distally, reflecting the direction of their growth (FIG. 1b). The number of LacZ⁺ streaks emanating from the distal part of the matrix and the bed decreased significantly by 3 months post labeling (FIG. 1d). In contrast, the streaks emerging from the proximal matrix persisted for at least 5 months (FIG. 1b, d). These streaks included the proximal matrix, distal matrix and bed cells (FIG. 1e). The progeny of proximal matrix and distal matrix both migrated vertically to give rise to individual keratinized layers of the nail plate¹⁰. These results, therefore, demonstrate that the proximal matrix comprises self-renewing NSCs that can sustain nail growth. Proximal matrix cells containing NSCs were, moreover, identified as highly proliferative (Ki67^{high}) and expressed K17 in addition to K14 (Supplementary FIG. S3). Isolated proximal matrix cells, enriched for K14⁺K17⁺ expression (FIG. 1f, g), exhibited the highest colony forming ability in vitro, a general characteristic of epithelial stem cells (FIG. 1 h-j).

Microarray analyses of proximal matrix versus distal matrix revealed that proximal matrix cells enriched with NSCs downregulated Wnt signaling pathway genes, which pathway is known to regulate embryonic development of limb/nail organ¹²⁻¹⁴ as well as differentiation of epithelial and melanocyte stem cells¹⁵. Analyses with Wnt reporter mice showed that Axin2-LacZ signal started from the distal part of the K17⁺ NSC region and persisted into the distal matrix, whereas the TOPGAL signal was seen in the K17⁻ distal matrix^{17,18}. Although these two markers distribute differently¹⁶, both signals were absent in the proximal end of the nail matrix (Supplementary FIG. S5). Additionally, TCF1, a nuclear mediator of Wnt signaling¹⁹, and Wntless (Wls), required for Wnt ligand secretion²⁰, were missing in the proximal end of the matrix. Moreover, several keratins that contained a Tcf-1/Lef-1 consensus binding site were upregulated in the distal matrix relative to that of the NSC region (Supplementary Table 1)^{21,22}.

K14-CreER;β-catenin^{fl/fl} (cKO) mice, wherein β-catenin, an essential mediator of Wnt signaling, is deleted in adult epithelium, were used to verify the role of Wnt activation in the nail epithelium (FIG. 2a). At two months after induction of β-catenin deletion by TAM treatment, nail formation is abrogated (FIG. 2b-e), as revealed by the lack of AE13, a marker for keratinized nail cells²² (FIG. 2f). It is also intriguing to note that the entire nail epithelium exhibits characteristics of the NSC region (K17⁺Ki67^{high}) (FIG. 2g-i) in the absence of β-catenin. This finding suggests that modulation of Wnt signaling can be used advantageously to modulate NSC cell populations.

To investigate how nail differentiation is linked to digit regeneration upon amputation, the present inventors treated cKO mice with TAM beginning immediately after digit amputation (FIG. 3a). In control mice, the nail resumed its original structure by 5 weeks (FIG. 3b) and the amputated digit bone regenerated along with nail regeneration (FIG. 3c-f). In cKO mice, the nail failed to regenerate (FIG. 3b, e). Bone regeneration in these mice was also completely blocked (FIG. 3c, d, f). Intact non-amputated digits in cKO mice (internal control) maintained similar digit bone length compared with intact digits in control mice at 5 weeks after TAM treatment (FIG. 3f).

Although time-course studies demonstrated that β-catenin was depleted in nail epithelial cells of cKO mice by one week after TAM induction (Supplementary FIG. S7), the amputated areas of both control and cKO mice were similarly re-epithelialized two weeks after amputation. In control mice, the regenerating nail matrix displayed Wnt activation with TOPGAL activity (FIG. 3g), contiguous with the original nail matrix cells, which permitted nail differentiation. Underneath the Wnt-active regenerating matrix, mesenchymal cells were actively proliferating (FIG. 3i). The majority (about 90%) of these proliferating cells were found to express Runx2²³, a marker for osteoblast commitment (Supplementary FIG. S8). In cKO mice, however, Runx2⁺ progenitors and Sp7+ osteoblasts were not induced to proliferate, and the expression of Bmp4, functionally critical for digit bone regeneration⁸, was missing in cKO digits (Supplementary FIG. S8). Nerves that are vital for regeneration of rodent digits²⁴ and amphibian limbs²⁵ are, moreover, located in the proliferative Runx2⁺ mesenchyme close to the Wnt-active nail epithelial cells in control mice, whereas nerves did not extend to the regeneration area close to the epithelium in cKO mice (FIG. 1h, Supplementary FIG. S9). Semaphorin 5a (Sema5a), an axon guidance molecule²⁶, is upregulated in control nail epithelium at 3 weeks after amputation, but not in that of cKO (Supplementary FIG. S10).

Additional results presented herein reveal that Wnt activation in the nail epithelium performs dual functions to promote both nail regeneration and Runx2⁺ mesenchymal cell growth through its ability to induce nerve-dependent FGF2 expression. See, for example, Supplementary FIGS. S11, S12, S13, and S14.

To address why digits do not regenerate after amputations proximal to the nail (Supplementary FIG. S2), the present inventors performed additional experiments. Careful examination of the amputated digits showed that amputations of the visible nail plate (i.e., removal of more than 50% of distal phalanx) do not remove the entire NSC region although it is known to fail in regeneration¹⁰ (Supplementary FIG. S15). Unlike distal amputations that induce regeneration, these amputations within the NSC region removed the distal matrix expressing Wntless which is required for initiation of Wnt signaling (Supplementary FIG. S15). Consequently, these amputations failed to activate epithelial Wnt signaling, as revealed by the lack of nuclear β-catenin and TCF1 expression after re-epithelialization (FIG. 4b-c), resulting in the failure to regenerate the nail and digit (FIG. 4g, Supplementary FIG. S2).

To test whether stabilization of β-catenin in K14+ epithelium, including the NSC region, can induce digit regeneration, the present inventors treated K14-CreER;β-catenin^{fl/ex3} mice with TAM following completion of re-epithelialization (FIG. 4a). One week after the initial TAM treatment, basal nail epithelial cells including NSC region exhibited nuclear β-catenin (FIG. 4b). In these tissues, NSC progeny expressed TCF1 as they regenerate distal matrix whereas the proximal end of the NSC region contained TCF1 negative cells (FIG. 4c). Regeneration of TCF1⁺ distal nail matrix in these mutant mice accompanied the formation of a well-innervated blastema, which is never observed in control mice upon amputation at this proximal level (FIG. 4d). Nail epithelial FGF2 expression and proliferating Runx2+mesenchymal cells were also observed, which in turn led to digit bone regeneration (FIG. 4e-f, Supplementary FIG. S16). In these mice, nail regeneration was also grossly apparent and nails without amputations did not show any detectable changes (FIG. 4g, 4i, Supplementary FIG. 17).

### Example II

Cellular therapy in regenerative medicine holds great promise to improve organ function that is compromised by age, disease or trauma. Allogeneic keratinocytes and fibroblasts are routinely used to aid reepithelialization of open wounds in human patients (Kirsner et al., 2012). Adult epithelial stem cells have, in particular, emerged as an efficient and robust source of cells to reconstitute normal structure and function to lost tissue (Sun and Lavker 2004, Pellegrini et al., 1997). Building on the potential presented by results described herein, an analogous approach using NSCs to promote digit regeneration is set forth herein. It is particularly directed to those amputations that extend beyond the nail level.

Further to the above, Wnt-active NSCs will be evaluated to determine if they can promote blastema cell proliferation in vitro using a co-culture system. To this end, blastema cells will be generated in vivo by digit tip amputation. Wnt-active or Wnt-inactive NSCs (negative control) will be co-cultured with these isolated blastema cells using a transwell insert. The proliferation rate of blastema cells in the presence of Wnt-active NSCs and Wnt-inactive NSCs will then be determined using, for example, Ki67 immunocytochemistry. To test whether paracrine factor(s) secreted by cultured Wnt-active NSCs and Wnt-inactive NSCs can promote digit regeneration in vivo, beads soaked with conditioned media from Wnt-active NSC and Wnt-inactive NSC cultures will be transplanted and assessed with respect to the extent of induced digit regeneration as described above. If Wnt-active NSCs secrete “regeneration promoting factors”, conditioned media obtained from culture of these cells will enhance proliferation of blastema cells in vitro. Injection of conditioned media containing “regeneration promoting factors” in amputation sites may induce blastema development and growth and eventual regrowth of digit bone. These results will reveal whether Wnt-active NSCs can serve as a signaling center capable of directing mesenchymal cells to direct regeneration.

In an experimental approach directed toward an in vivo animal model system, Wnt-active NSCs and Wnt-inactive NSCs (negative control) will be isolated. In one approach directed to this objective, the present inventors propose to delete or stabilize β-catenin in epithelial cells, using K14-creER;β-catenin^{fl/fl} and K14-creER;β-catenin^{(EX3)fl/+} mice, respectively. Double transgenic mice will either deplete or stabilize β-catenin in NSCs and their progeny upon tamoxifen (TAM) treatment. Double transgenic mice will be injected daily with TAM (1 mg/50 μl corn oil/mouse) intraperitoneally for 7 days starting at P20 to induce Cre activity, after which the mice will be sacrificed and NSCs isolated utilizing established methods involving microdissection and enzymatic digestion as described herein above.

Immunodeficient mice will be used as recipients. These mice will be amputated at the level proximal to the NSC niche. Upon completion of wound closure, NSCs (1×10⁴ cells) will be transplanted underneath the wound epidermis. Digits will be harvested 3 and 5 weeks after amputation and these samples will be utilized for tissue section analyses and whole mount alizarin red analyses. Initially, tissue sections will be utilized to determine if Wnt-active NSCs undergo growth and differentiation to form nail. To this end, the samples will be analyzed immunohistochemically with differentiation markers of nail epithelium including K14, K17 (Fleckman et al., 2013) and AE13 (Lynch et al., 1986). The degree of innervation, FGF2 expression, mesenchymal cell proliferation and digit bone regeneration will be assessed as described herein.

Conditioned media prepared from cultured Wnt-active NSCs and Wnt-inactive NSCs (negative control) will also be evaluated to determine if such media can serve as a surrogate for NSCs.

Experiments proposed herein provide a foundation for translating findings from results presented herein to much-needed clinical applications in mammalian digit/limb regeneration. It is noteworthy that recent pathological characterizations of the human nail unit suggest the presence of human NSCs (Sellheyer, 2013, J Dtsch Dermatol Ges 11(3):235-9; Sellheyer and Nelson, 2013, J Cutan Pathol 40:463-471; the entire content of each of which is incorporated herein by reference). Accordingly, the proposed experiments will further support the exploitation of NSCs in treatment regimens for amputees and will represent a significant advancement of regenerative medicine. Additionally, investigating how NSCs behave upon transplantation in other mammals will provide an important initiative to utilize NSCs in the treatment of irreversible nail dystrophies such as lichen planus and psoriasis (Holzberg, 2006, Dermatol Clin 24:349-354).

While certain of the particular embodiments of the present invention have been described and specifically exemplified above, it is not intended that the invention be limited to such embodiments. Various modifications may be made thereto without departing from the scope and spirit of the present invention, as set forth in the following claims.

### REFERENCES

1 Douglas, B. S. Conservative management of guillotine amputation of the finger in children. Aust Paediatr J 8, 86-89 (1972).
2 Borgens, R. B. Mice regrow the tips of their foretoes. Science 217, 747-750 (1982).
3 Zhao, W. & Neufeld, D. A. Bone regrowth in young mice stimulated by nail organ. J Exp Zool 271, 155-159, doi:10.1002/jez. 1402710212 (1995).
4 Mohammad, K. S., Day, F. A. & Neufeld, D. A. Bone growth is induced by nail transplantation in amputated proximal phalanges. Calcif Tissue Int 65, 408-410 (1999).
5 Rinkevich, Y., Lindau, P., Ueno, H., Longaker, M. T. & Weissman, I. L. Germ-layer and lineage-restricted stem/progenitors regenerate the mouse digit tip. Nature 476, 409-413, doi:10.1038/nature 10346 (2011).
6 Lehoczky, J. A., Robert, B. & Tabin, C. J. Mouse digit tip regeneration is mediated by fate-restricted progenitor cells. Proc Natl Acad Sci USA 108, 20609-20614, doi:10.1073/pnas. 1118017108 (2011).
7 Neufeld, D. A. Partial blastema formation after amputation in adult mice. J Exp Zool 212, 31-36, doi:10.1002/jez. 1402120105 (1980).
8 Han, M., Yang, X., Lee, J., Allan, C. H. & Muneoka, K. Development and regeneration of the neonatal digit tip in mice. Dev Biol 315, 125-135, doi:10.1016/j.ydbio.2007.12.025 (2008).
9 Neufeld, D. A. & Zhao, W. Phalangeal regrowth in rodents: postamputational bone regrowth depends upon the level of amputation. Prog Clin Biol Res 383A, 243-252 (1993).
10 Norton, L. A. Incorporation of thymidine-methyl-H3 and glycine-2-H3 in the nail matrix and bed of humans. J Invest Dermatol 56, 61-68 (1971).
11 Fleckman P, Jaeger K, Silva K A, Sundberg J P. Comparative anatomy of mouse and human nail units. Anat Rec (Hoboken) 296, 521-532 (2013)
12 Al-Qattan, M. M. WNT pathways and upper limb anomalies. J Hand Surg Eur Vol 36, 9-22, doi:10.1177/1753193410380502 (2011).
13 Blaydon, D. C. et al. The gene encoding R-spondin 4 (RSPO4), a secreted protein implicated in Wnt signaling, is mutated in inherited anonychia. Nat Genet. 38, 1245-1247, doi:10.1038/ng1883 (2006).
14 Adaimy, L. et al. Mutation in WNT10A is associated with an autosomal recessive ectodermal dysplasia: the odonto-onycho-dermal dysplasia. Am J Hum Genet. 81, 821-828, doi:10.1086/520064 (2007).
15 Rabbani, P. et al. Coordinated activation of Wnt in epithelial and melanocyte stem cells initiates pigmented hair regeneration. Cell 145, 941-955, doi:10.1016/j.cell.2011.05.004 (2011).
16 Al Alam, D. et al. Contrasting expression of canonical Wnt signaling reporters TOPGAL, BATGAL and Axin2 (LacZ) during murine lung development and repair. PLoS One 6, e23139, doi:10.1371/journal.pone.0023139 (2011).
17 Lin, M. H. & Kopan, R. Long-range, nonautonomous effects of activated Notch1 on tissue homeostasis in the nail. Dev Biol 263, 343-359 (2003).
18 Nakamura, M. & Ishikawa, O. The localization of label-retaining cells in mouse nails. J Invest Dermatol 128, 728-730, doi:10.1038/sj.jid.5701062 (2008).
19 van de Wetering, M. et al. Armadillo coactivates transcription driven by the product of the Drosophila segment polarity gene dTCF. Cell 88, 789-799 (1997).
20 Banziger, C. et al. Wntless, a conserved membrane protein dedicated to the secretion of Wnt proteins from signaling cells. Cell 125, 509-522, doi:10.1016/j.cell.2006.02.049 (2006).
21 Zhou, P., Byrne, C., Jacobs, J. & Fuchs, E. Lymphoid enhancer factor 1 directs hair follicle patterning and epithelial cell fate. Genes Dev 9, 700-713 (1995).
22 Lynch, M. H., O'Guin, W. M., Hardy, C., Mak, L. & Sun, T. T. Acidic and basic hair/nail (“hard”) keratins: their colocalization in upper cortical and cuticle cells of the human hair follicle and their relationship to “soft” keratins. J Cell Biol 103, 2593-2606 (1986).
23 Ducy, P., Zhang, R., Geoffroy, V., Ridall, A. L. & Karsenty, G. Osf2/Cbfa1: a transcriptional activator of osteoblast differentiation. Cell 89, 747-754 (1997).
24 Mohammad, K. S. & Neufeld, D. A. Denervation retards but does not prevent toetip regeneration. Wound Repair Regen 8, 277-281 (2000).
25 Brockes, J. P. The nerve dependence of amphibian limb regeneration. J Exp Biol 132, 79-91 (1987).
26 Kantor, D. B. et al. Semaphorin 5A is a bifunctional axon guidance cue regulated by heparan and chondroitin sulfate proteoglycans. Neuron 44, 961-975, doi:10.1016/j.neuron.2004.12.002 (2004).
27 Zhang, Y. et al. Activation of beta-catenin signaling programs embryonic epidermis to hair follicle fate. Development 135, 2161-2172, doi:10.1242/dev.017459 (2008).
28 Mullen, L. M., Bryant, S. V., Torok, M. A., Blumberg, B. & Gardiner, D. M. Nerve dependency of regeneration: the role of Distal-less and FGF signaling in amphibian limb regeneration. Development 122, 3487-3497 (1996).
29 Kawakami, Y. et al. Wnt/beta-catenin signaling regulates vertebrate limb regeneration. Genes Dev 20, 3232-3237, doi:10.1101/gad.1475106 (2006).
30 Yokoyama, H., Ogino, H., Stoick-Cooper, C. L., Grainger, R. M. & Moon, R. T. Wnt/beta-catenin signaling has an essential role in the initiation of limb regeneration. Dev Biol 306, 170-178, doi:10.1016/j.ydbio.2007.03.014 (2007).
31 Harada, N. et al. Intestinal polyposis in mice with a dominant stable mutation of the beta-catenin gene. Embo J 18, 5931-5942, doi:10.1093/emboj/18.21.5931 (1999).
32 Vasioukhin, V., Degenstein, L., Wise, B. & Fuchs, E. The magical touch: genome targeting in epidermal stem cells induced by tamoxifen application to mouse skin. Proc Natl Acad Sci USA 96, 8551-8556 (1999).
33 Lowry, W. E. et al. Defining the impact of beta-catenin/Tcf transactivation on epithelial stem cells. Genes Dev 19, 1596-1611, doi:10.1101/gad.1324905 (2005).
34 Myung, P. S., Takeo, M., Ito, M. & Atit, R. P. Epithelial Wnt Ligand Secretion Is Required for Adult Hair Follicle Growth and Regeneration. J Invest Dermatol, doi:10.1038/jid.2012.230 (2012).
35 DasGupta, R. & Fuchs, E. Multiple roles for activated LEF/TCF transcription complexes during hair follicle development and differentiation. Development 126, 4557-4568 (1999).
36 Lustig, B. et al. Negative feedback loop of Wnt signaling through upregulation of conductin/axin2 in colorectal and liver tumors. Mol Cell Biol 22, 1184-1193 (2002).
37. Y. Barrandon, H. Green, Three clonal types of keratinocyte with different capacities for multiplication. Proc Natl Acad Sci USA 84:2302-2306 (1987).

1. A method for promoting tissue regeneration in a subject with an amputation that removed a body part leaving a truncated body part attached to the subject, the method comprising administering to a distal tip of the truncated body part a cell population comprising nail stem cells contacted with at least one activator of a Wnt signaling pathway, wherein the administering promotes tissue regeneration in the subject, thereby restoring part or all of the amputated body part.

2. The method of claim 1, wherein the body part amputated comprises one, two, or three digits of a finger or toe, a finger, a toe, or a limb or a portion thereof.

3. The method of claim 1, wherein the cell population comprising nail stem cells is isolated from the proximal nail matrix.

4. The method of claim 1, wherein the cell population comprising nail stem cells comprises greater than 75%, 80%, 85%, 90%, 95%, 96%, 97%, 98%, or 99% K14+/K17+ double positive nail stem cells or comprises 100% K14+/K17+ double positive nail stem cells.

5. The method of claim 1, wherein the cell population comprising nail stem cells is expanded in vitro prior to administering.

6. (canceled)

7. The method of claim 1, wherein the cell population comprising nail stem cells consists of isolated proximal nail matrix cells.

8. The method of claim 7, wherein the isolated proximal nail matrix cells exclude cells of the distal nail matrix.

9. (canceled)

10. The method of claim 1, wherein the cell population comprising nail stem cells is isolated from the subject or from an allogeneic subject.

11. The method of claim 1, wherein the at least one activator of a Wnt signaling pathway is Wnt3, Wnt7a, Wnt7b, or Wnt10a.

12. The method of claim 1, wherein the nail stem cells are contacted with the at least one activator of a Wnt signaling pathway before, during, and/or after the administering.

13. (canceled)

14. The method of claim 1, wherein the amputated body part is a digit tip.

15. The method of claim 1, wherein the subject is a mammal.

16. The method of claim 14, wherein the subject is a human.

17. A method for promoting digit tip regeneration in a subject with an amputation that removed a digit tip leaving a truncated finger or toe attached to the subject, the method comprising administering to a distal tip of the truncated finger or toe a cell population comprising nail stem cells contacted with at least one activator of a Wnt signaling pathway, wherein the administering promotes digit tip regeneration in the subject, thereby restoring part or all of the amputated digit tip.

18-30. (canceled)

31. A method for expanding a population of nail stem cells in vitro and/or generating a population of cells having multipotent regenerative capacity from isolated nail stem cells in vitro, the method comprising isolating tissue comprising nail stem cells from a subject, culturing the nail stem cells to expand the population of nail stem cells in vitro, and activating Wnt signaling in the expanded population of nail stem cells, wherein the culturing induces proliferation of the nail stem cells and the activating promotes differentiative capacity of the nail stem cells, thereby expanding the population of nail stem cells and/or generating the population of cells having multipotent regenerative capacity.

32. The method of claim 31, wherein the tissue comprising nail stem cells is nail matrix.

33. The method of claim 32, wherein the nail matrix consists of proximal nail matrix.

34. The method of claim 31, wherein the activating is achieved by contacting the nail stem cells with at least one activator of a Wnt signaling pathway or by transfecting/transducing the nail stem cells with a vector comprising a nucleic acid sequence encoding an exogenous polypeptide that activates a Wnt signaling pathway in transduced nail stem cells.

35-40. (canceled)

41. The method of claim 31, further comprising administering the expanded population of nail stem cells or the population of cells having multipotent regenerative capacity to a recipient in need thereof.

42. (canceled)

43. The method of claim 41, wherein the recipient in need thereof is afflicted with nail dystrophy or nail psoriasis.

44-53. (canceled)
